SEQUENCE LISTING (14 sequences)

SEQ ID NO: 1 - DNA, 24 bp, Artificial
  F1 PCR PRIMER
gggcaagttgctcagctgtttttg

SEQ ID NO: 2 - DNA, 23 bp, ARTIFICIAL
  R1 PCR PRIMER
tccgcatggtttagcaggattca

SEQ ID NO: 3 - DNA, 24 bp, ARTIFICIAL
  F1 PCR PRIMER
atcgaacctgggtcttctgcattg

SEQ ID NO: 4 - DNA, 23 bp, ARTIFICIAL
  R1 PCR PRIMER
tccgcatggtttagcaggattca

SEQ ID NO: 5 - DNA, 25 bp, ARTIFICIAL
  P PCR PRIMER
acgtactcttcatttcacagcctac

SEQ ID NO: 6 - DNA, 25 bp, ARTIFICIAL
  F2 PCR PRIMER
gtctggggtgagatagttttcttgg

SEQ ID NO: 7 - DNA, 24 bp, ARTIFICIAL
  R2 PCR PRIMER
ggcagagatgttggtcttgggtgt

SEQ ID NO: 8 - DNA, 10 bp, Bos taurus
gacggtggtt

SEQ ID NO: 9 - DNA, 11 bp, BOS TAURUS
tgtatcccacc

SEQ ID NO: 10 - DNA, 11 bp, BOS TAURUS
tgtatcccgcc

SEQ ID NO: 11 - DNA, 11 bp, BOS TAURUS
tgtagcccacc

SEQ ID NO: 12 - DNA, 11 bp, BOS TAURUS
tgtagcccgcc

SEQ ID NO: 13 - DNA, 11 bp, BOS TAURUS
tgtagctgacc

SEQ ID NO: 14 - DNA, 11 bp, BOS TAURUS
tgtatcccgtt

(19) United States
(12) Patent Application Publication
(10) Pub. No.: US 2017/0058362 A1
(43) Pub. Date: Mar. 2, 2017

(54) METHOD OF IDENTIFYING THE PRESENCE OF FOREIGN ALLELES IN A DESIRED HAPLOTYPE

(71) Applicant: Recombinetics, Inc., Saint Paul, MN (US)

(72) Inventors: Scott C. Fahrenkrug, Minneapolis, MN (US); Tad S. Sonstegard, Centreville, MD (US); Daniel F. Carlson, Woodbury, MN (US)

(21) Appl. No.: 15/253,150

(22) Filed: Aug. 31, 2016

Related U.S. Application Data
(60) Provisional application No. 62/212,840, filed on Sep. 1, 2015.
(60) Provisional application No. 62/321,942, filed on Apr. 13, 2016.

(51) Int. Cl. C12Q 1/68; A01K 67/027

(52) U.S. Cl. CPC C12Q 1/6888; A01K 67/0275; C12Q 2600/124; C12Q 2600/172; C12Q 2600/156; A01K 2217/15; A01K 2227/101; C12Q 2600/16

(57) ABSTRACT

Methods and kits to determine the presence of exogenous alleles within a native haplotype are provided. Introduction of foreign alleles into livestock genomes has provided the ability to introduce specific desirable traits. The present disclosure provides methods to identify the presence of exogenous alleles that foreign to a haplotype at a target locus, and identify specific markers that are native to the haplotype. Identification of exogenous genes at a target locus, flanked by native markers is indicative that the exogenous gene is present through molecular engineering. Conversely, the presence of an exogenous gene that are only partially flanked by native markers is indicative that the allele is present due to sexual breeding.

### CROSS REFERENCE TO RELATED APPLICATIONS

This patent applications claims priority to U.S. Provisional Applications 62/212,840 filed Sep. 1, 2015 and 62/321,942 filed Apr. 13, 2016 which are both hereby incorporated by reference in their entirety.

### FIELD OF THE DISCLOSURE

The disclosure is directed to methods and kits for the detection of target alleles inserted in a desired haplotype.

### BACKGROUND OF THE DISCLOSURE

Livestock and other animals have been domesticated by man since the beginnings of civilization. Reasons for man's domestication of animals include for food, clothing, protection, resistance to disease and companionship. Particular breeds of livestock have been developed having desirable traits, or lacking undesirable or dangerous ones. For example cattle have been bred for many reasons including meat production, quality of leather, docility and even aggressiveness (think bullfighting). Similarly, horses have been bred to have various traits including size and strength (draft horses), speed (thoroughbreds), stamina and heat tolerance (Arabians) and general disposition, speed and hardiness (quarter horses) desirable for specific uses. Similarly, all other livestock animals have been “inbred” to some extent, either to obtain a desirable trait from a parent or ancestor or to exclude undesirable. In each case, sexual reproduction of animal requires mixing of parent genes such that many traits from both parents are imparted to the offspring.

All methods of breeding animals, by definition, require sexual reproduction. In sexual reproduction, the diploid gametogenic cells of the male and a female gonads each carrying a single set of chromosomes from its mother and father (as do all other cells), undergo reduction and division in meiosis I. During meiosis I the chromosomes duplicate (4n) and crossover between homologous chromosomes may occur resulting in new sets of genetic information within each chromosome. Meiosis I is followed by two phases of cell division resulting in four haploid gametes each carrying a unique set of genetic information. Because genetic recombination results in new gene sequences or combinations of genes, diversity is increased.

However, in livestock animals, carefully bred over hundreds if not thousands of years, increased diversity is not desirable. What is desirable is that the animal exemplifies and maintains the traits for which it has been bred. For example, there are over 800 breeds of cattle worldwide. Some cattle are bred for suitability for a particular climate however, the most numerous are bred for particular agricultural purposes including milk production and/or beef production or for draft purposes. For example, Herefords are primarily bred for meat while Holsteins are primarily dairy animals and Simmental cattle are bred for both meat and dairy purposes. Further, such breeds also have predictable, non-metric traits such as temperament and reproductive capacity. It will be appreciated that most livestock animals, including horses, sheep, pigs etc. similarly express specific traits that are considered dependable and desirable and to exclude undesirable traits such as aggressiveness or susceptibility to disease that are undesirable.

In the desire to install beneficial traits in animals, livestock, has been bred over thousands of years to include particularly desirable traits in a single breed. Generally, animal breeds are a specific group of domestic animals having homogenous appearance (phenotype), homogenous behavior, and/or other characteristics that distinguish them from other animals of their species and result from selective breeding. Selective breeding requires sexually mating individuals with desired traits with other individuals to breed “true” for the desired trait and for the traits of the breed as a whole. Animal breeding to develop desirable breeds and a desirable traits in those breeds has a long established science requiring inbreeding, linebreeding, and outcrossing. However, during the many generations needed to develop desirable breeds of livestock, the actual genetic diversity of a breed has become greatly reduced even though its actual numbers may be very large. Consequently, the effective population size (Ne) has become extremely small for any particular breed. For example, Hayes et al. (U.S. Pub No. 2014/0220575, hereby incorporated in its entirety), estimate that, among cattle, the Ne of Holstein-Friesians is estimated to be between 50 and 100; Brown Swiss about 46; Holstein 49 and Danish Red 47. Among sheep, 35 for Dorset-Rarnboulliet-Finnsheep cross. Pigs have a slightly higher Ne estimated at <200 for Harmegnies; 85 for Duroc/Large white and 300 for Large white. Chicken show similarly small Ne for breeds such as Layers which are estimated to be between 91 and 123. This low genetic diversity means that the physical characteristics of each breed are well recognized and that the chance of an undesirable characteristic occurring in a widely used breed is extremely low and generally limited to the occurrence of spontaneous mutations.

Consequently, the ability to introduce new traits in various livestock breeds is limited and time-consuming. For example, introduction of a new trait into a desirable domestic breed requires crossbreeding an animal having a desired trait with a high-quality female of the desired breed, selection of phenotypically acceptable progeny of the desired breed having the desired trait it was crossbred for and backcrossing positive progeny with further males or females of the desired breed to arrive at an animal having all the characteristics of the breed but also including the desired trait while removing all other traits from the out breed parent. Consequently, introduction of a new trait into a valued breed takes many generations of breeding to provide a stably introduced trait into an animal but lacking all other qualities of the introduced species and breeding true for all other characteristics of the valued breed.

Breeding of valuable animals requires many generations of selective breeding to stably introduce a single trait and backcrossing the progeny to provide an animal which in all other characteristics are consistent with the well-recognized traits of the breed. Therefore, there is a need for easier ways to introduce desirable traits in livestock animals and to test those animals to determine whether novel traits they may express are the result of sexual reproduction or the result of the directed introduction of specific traits carried by specific genes within the genotype of desired livestock breed.

Recently, new techniques of gene modification and animal cloning has provided the ability to introduce specific genes or alleles into an animals genomes such as by gene editing, (see for example, U.S. Pub Nos 2014033807, 20140201857, 20140041066, 20130117870 and 20130090522 to Recombinetics, Inc., each of which is incorporated by reference hereby in its entirety). These techniques allow for the introduction of novel traits directly into the genome of valuable breeds of animals without the risk of adding detrimental or undesirable traits into that breed and within less than a generation of the animal.

Currently, valuable breeds of livestock are entered into a breed registry or studbook that lists each animal's pedigree and can be followed for many generations. This written registry has allowed animal breeders to identify valuable and prolific female and male stock and to identify and avoid breeding with undesirable animals. However, the presentation of animals of a known breed with new traits requires the animal breeder to identify how the new trait was introduced such as by planned crossing with a known breed or an unanticipated sexual occurrence with an unknown or undesirable animal. Thus, it would be helpful to identify techniques and/or methods which would help identify the source of new traits in a particular breed of livestock.

### SUMMARY OF THE DISCLOSURE

The present disclosure provides methods and kits to identify an animal that is the product of genetic manipulation to introduce a foreign or exogenous allele (genes) at a target locus of a livestock animal while maintaining the native genome of the host animal.

Therefore, in one exemplary embodiment, the disclosure provides a method to identify an animal that has an exogenous allele inserted at a target locus and that has genetic markers within a native haplotype containing the target locus. In various embodiments, the disclosure further comprises identifying the exogenous allele using Southern hybridization, in situ hybridization, PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP). In various exemplary embodiments, indentifying the native markers comprises using Southern hybridization, in situ hybridization, PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP). In various exemplary embodiments, the markers are within 500 bp of the target locus. In various other exemplary embodiments there are at least 5 markers. In other exemplary embodiments there are at least 4 markers, 3 markers or two markers. In other exemplary embodiments, at least two of the markers flank the target locus. In these exemplary embodiments, by detecting the presence of an exogenous allele at a target locus within a native haplotype, the presence of the exogenous allele due to breeding with another animal can be excluded while the presence of an exogenous allele at a target locus within a native haplotype is decisive in identifying an animal that resulting from genetic modification.

In yet another exemplary embodiment, the disclosure provides a kit for determining whether an animal is the product of sexual breeding or is the product of genetic modification. In this exemplary embodiment, the kit comprises two or more probes and/or primers for a known haplotype. In addition, in this exemplary embodiment, the kit also includes one or more probes or primers for an allele, located at a target locus that is foreign to the haplotype. In various exemplary embodiments, the kit also comes with instructions for use. In yet other exemplary embodiments, the kit further is contained in a container. In yet other embodiments, the kit further comes with reagents, ampules or test tubes required for determining whether the animal is a product of sexual breeding or genetic modification. In various exemplary embodiments, the kit includes a mailing label.

These and other features and advantages of the present disclosure will be set forth or will become more fully apparent in the description that follows and in the appended claims. The features and advantages may be realized and obtained by means of the instruments and combinations particularly pointed out in the appended claims. Furthermore, the features and advantages of the disclosure may be learned by the practice of the methods and techniques disclosed herein or will be apparent from the description, as set forth hereinafter.

### BRIEF DESCRIPTION OF THE DRAWINGS

Various exemplary embodiments of the compositions and methods according to the disclosure will be described in detail, with reference to the following figures wherein:

FIG. 1, TALEN-mediated introgression of Polled. (A) Strategy for introgressing the Pc allele into Holstein (HORNED) cells. The Pc allele is a tandem repeat of 212 bp (red arrow) with a 10-bp deletion (not shown). TALENs were developed to specifically target the HORNED allele (green vertical arrow), which could be repaired by homologous recombination using the Pc HDR plasmid. Primer sets used in B are depicted. (B) Representative images of colonies with homozygous or heterozygous introgression of Pc. Three primer sets, indicated by number, were used for positive classification of candidate colonies: set 1, F1+R1; set 2, F2+R2; and set 3, F1+P (Pc-specific). Amplicons generated using positive control templates (P, plasmid template containing a sequence-verified Pc 1,748-bp insert between primers F1 and R1; H, Holstein bull genomic DNA) are shown. The identity of the PCR products was confirmed by sequencing of F1+R1 amplicons.

FIG. 2 is a schematic illustrating crossing over during meiotic recombination.

FIG. 3, is a schematic illustrating the genetic identification of an introduced allele either by recombination, spontaneous mutation or non-meiotic introgression.

### DETAILED DESCRIPTION OF THE EXEMPLARY EMBODIMENTS

The present disclosure provides methods and kits to identify an animal that is the product of genetic manipulation to introduce a foreign or exogenous allele at a target locus while maintaining the native genome of the host animal.

Unless defined otherwise, all technical and scientific terms used herein have the same meanings as commonly understood by one of ordinary skill in the art to which this disclosure belongs. All publications and patents specifically mentioned herein are incorporated by reference for all purposes including describing and disclosing the chemicals, instruments, statistical analyses and methodologies which are reported in the publications which might be used in connection with the disclosure. All references cited in this specification are to be taken as indicative of the level of skill in the art. Nothing herein is to be construed as an admission that the disclosure is not entitled to antedate such disclosure by virtue of prior invention.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

“Additive Genetic Effects” as used herein means average individual gene effects that can be transmitted from parent to progeny.

“Allele” as used herein refers to an alternate form of a gene. It also can be thought of as variations of DNA sequence. For instance if an animal has the genotype for a specific gene of Bb, then both B and b are alleles.

“DNA Marker” refers to a specific DNA variation that can be tested for association with a physical characteristic.

“Genotype” refers to the genetic makeup of an animal.

“Genotyping (DNA marker testing)” refers to the process by which an animal is tested to determine the particular alleles it is carrying for a specific genetic test.

“Simple Traits” refers to traits such as coat color and horned status and some diseases that are carried by a single gene.

“Complex Traits” refers to traits such as reproduction, growth and carcass that are controlled by numerous genes.

“Complex allele”—coding region that has more than one mutation within it. This makes it more difficult to determine the effect of a given mutation because researchers cannot be sure which mutation within the allele is causing the effect.

“Copy number variation” (CNVs) a form of structural variation—are alterations of the DNA of a genome that results in the cell having an abnormal or, for certain genes, a normal variation in the number of copies of one or more sections of the DNA. CNVs correspond to relatively large regions of the genome that have been deleted (fewer than the normal number) or duplicated (more than the normal number) on certain chromosomes. For example, the chromosome that normally has sections in order as A-B-C-D might instead have sections A-B-C-“Repetitive element” patterns of nucleic acids (DNA or RNA) that occur in multiple copies throughout the genome. Repetitive DNA was first detected because of its rapid reassociation kinetics.

“Quantitative variation” variation measured on a continuum (e.g. height in human beings) rather than in discrete units or categories. See continuous variation. The existence of a range of phenotypes for a specific character, differing by degree rather than by distinct qualitative differences.

“Homozygous” refers to having two copies of the same allele for a single gene such as BB.

“Heterozygous” refers to having different copies of alleles for a single gene such as Bb.”

“Locus” (plural “loci”) refers to the specific locations of a maker or a gene on a chromosome.

“Centimorgan (Cm)” a unit of recombinant frequency for measuring genetic linkage. It is defined as the distance between chromosome positions (also termed, loci or markers) for which the expected average number of intervening chromosomal crossovers in a single generation is 0.01. It is often used to infer distance along a chromosome. It is not a true physical distance however.

“Chromosomal crossover” (“crossing over”) is the exchange of genetic material between homologous chromosomes inherited by an individual from its mother and father. Each individual has a diploid set (two homologous chromosomes, e.g., 2n) one each inherited from its mother and father. During meiosis I the chromosomes duplicate (4n) and crossover between homologous regions of chromosomes received from the mother and father may occur resulting in new sets of genetic information within each chromosome. Meiosis I is followed by two phases of cell division resulting in four haploid gametes each carrying a unique set of genetic information. Because genetic recombination results in new gene sequences or combinations of genes, diversity is increased. Crossover usually occurs when homologous regions on homologous chromosomes break and then reconnect to the other chromosome.

“Marker Assisted Selection (MAS)” refers to the process by which DNA marker information is used to assist in making management decisions.

“Marker Panel” a combination of two or more DNA markers that are associated with a particular trait.

“Non-additive Genetic Effects” refers to effects such as dominance and epistasis. Codominance is the interaction of alleles at the same locus while epistasis is the interaction of alleles at different loci.

“Nucleotide” refers to a structural component of DNA that includes one of the four base chemicals: adenine (A), thymine (T), guanine (G), and cytosine (C).

“Phenotype” refers to the outward appearance of an animal that can be measured. Phenotypes are influenced by the genetic makeup of an animal and the environment.

“Single Nucleotide Polymorphism (SNP)” is a single nucleotide change in a DNA sequence.

“Haploid genotype” or “haplotype” refers to a combination of alleles, loci or DNA polymorphisms that are linked so as to cosegregate in a significant proportion of gametes during meiosis. The alleles of a haplotype may be in linkage disequilibrium (LD).

“Linkage disequilibrium (LD)” is the non-random association of alleles at different loci i.e. the presence of statistical associations between alleles at different loci that are different from what would be expected if alleles were independently, randomly sampled based on their individual allele frequencies. If there is no linkage disequilibrium between alleles at different loci they are said to be in linkage equilibrium.

The term “restriction fragment length polymorphism” or “RFLP” refers to any one of different DNA fragment lengths produced by restriction digestion of genomic DNA or cDNA with one or more endonuclease enzymes, wherein the fragment length vanes between individuals in a population.

“Introgression” also known as “introgressive hybridization”, is the movement of a gene or allele (gene flow) from one species into the gene pool of another by the repeated backcrossing of an interspecific hybrid with one of its parent species. Purposeful introgression is a long-term process; it may take many hybrid generations before the backcrossing occurs.

“Nonmeiotic introgression” genetic introgression via introduction of a gene or allele in a diploid (non-gemetic) cell. Non-meiotic introgression does not rely on sexual reproduction and does not require backcrossing and, significantly, is carried out in a single generation. In non-meiotic introgression an allele is introduced into a haplotype via homologous recombination. The allele may be introduced at the site of an existing allele to be edited from the genome or the allele can be introduced at any other desirable site.

“Transcription activator-like effector nucleases (TALENs)” are artificial restriction enzymes generated by fusing a TAL effector DNA-binding domain to a DNA cleavage domain.

“Indel” as used herein is shorthand for “insertion” or “deletion” referring to a modification of the DNA in an organism.

“Genetic marker” as used herein refers to a gene/allele or known DNA sequence with a known location on a chromosome. The markers may be any genetic marker e.g., one or more alleles, haplotypes, haplogroups, loci, quantitative trait loci, or DNA polymorphisms [restriction fragment length polymorphisms (RFLPs), amplified fragment length polymorphisms (AFLPs), single nuclear polymorphisms (SNPs), indels, short tandem repeats (STRs), microsatellites and minisatellites]. Conveniently, the markers are SNPs or STRs such as microsatellites, and more preferably SNPs. Preferably, the markers within each chromosome segment are in linkage disequilibrium.

As used herein the term “host animal” means an animal which has a native genetic complement of a recognized species or breed of animal.

As used herein, “native haplotype” or “native genome” means the natural DNA of a particular species or breed of animal that is chosen to be the recipient of a gene or allele that is not present in the host animal.

As used herein the term “genetic modification” refers to is the direct manipulation of an organism's genome using biotechnology.

As used herein the term “target locus” means a specific location of a known allele on a chromosome.

As used herein, the term “quantitative trait” refers to a trait that fits into discrete categories. Quantitative traits occur as a continuous range of variation such as that amount of milk a particular breed can give or the length of a tail. Generally, a larger group of genes controls quantitative traits.

As used herein, the term “qualitative trait” is used to refer to a trait that falls into different categories. These categories do not have any certain order. As a general rule, qualitative traits are monogenic, meaning the trait is influenced by a single gene. Examples of qualitative traits include blood type and flower color, for example.

As used herein, the term “quantitative trait locus (QTL)” is a section of DNA (the locus) that correlates with variation in a phenotype (the quantitative trait).

As used herein the term “cloning” means production of genetically identical organisms asexually.

“Somatic cell nuclear transfer” (“SCNT”) is one strategy for cloning a viable embryo from a body cell and an egg cell. The technique consists of taking an enucleated oocyte (egg cell) and implanting a donor nucleus from a somatic (body) cell.

“Orthologous” as used herein refers to a gene with similar function to a gene in an evolutionarily related species. The identification of orthologues is useful for gene function prediction. In the case of livestock, orthologous genes are found throughout the animal kingdom and those found in other mammals may be particularly useful for transgenic replacement. This is particularly true for animals of the same species, breed or lineages wherein species are defined two animals so closely related as to being able to produce fertile offspring via sexual reproduction; breed is defined as a specific group of domestic animals having homogenous phenotype, homogenous behavior and other characteristics that define the animal from others of the same species; and wherein lineage is defined as continuous line of descent; a series of organisms, populations, cells, or genes connected by ancestor/descendent relationships. For example domesticated cattle are of two distinct lineages both arising from ancient aurochs. One lineage descends from the domestication of aurochs in the Middle East while the second distinct lineage descends from the domestication of the aurochs on the Indian subcontinent.

“Genotyping” or “genetic testing” generally refers to detecting one or more markers of interest e.g., SNPs in a sample from an individual being tested, and analyzing the results obtained to determine the haplotype of the subject. As will be apparent from the disclosure herein, it is one exemplary embodiment to detect the one or more markers of interest using a high-throughput system comprising a solid support consisting essentially of or having nucleic acids of different sequence bound directly or indirectly thereto, wherein each nucleic acid of different sequence comprises a polymorphic genetic marker derived from an ancestor or founder that is representative of the current population and, more preferably wherein said high-throughput system comprises sufficient markers to be representative of the genome of the current population. Preferred samples for genotyping comprise nucleic acid, e.g., RNA or genomic DNA and preferably genomic DNA. A breed of livestock animal can be readily established by evaluating its genetic markers.

Livestock may be genotyped to identify various genetic markers. Genotyping is a term that refers to the process of determining differences in the genetic make-up (genotype) of an individual by determining the individual's DNA sequence using a biological assay and comparing it to another individual's sequence or to a reference sequence. A genetic marker is a known DNA sequence, with a known location on a chromosome; they are consistently passed on through breeding, so they can be traced through a pedigree or phylogeny. Genetic markers can be a sequence comprising a plurality of bases, or a single nucleotide polymorphism (SNP) at a known location. The breed of a livestock animal can be readily established by evaluating its genetic markers. Many markers are known and there are many different measurement techniques that attempt to correlate the markers to traits of interest, or to establish a genetic value of an animal for purposes of future breeding or expected value.

Genetic testing of animals can be performed using extremely small tissue samples, a hair follicle, for example, isolated from the tail of an animal to be tested can be used. Other examples of readily accessible samples include, for example, skin or a bodily fluid or an extract thereof or a fraction thereof. For example, a readily accessible bodily fluid includes, for example, whole blood, saliva, semen or urine. Exemplary whole blood fractions are selected from the group consisting of buffy-coat fraction, Fraction II+III obtainable by ethanol fractionation of Cohn (E. J. Cohn et al., J. Am. Chem. Soc., 68, 459 (1946), Fraction II obtainable by ethanol fractionation of Cohn (E. J. Cohn et al., J. Am. Chem. Soc., 68, 459 (1946), albumin fraction, an immunoglobulin-containing fraction and mixtures thereof, Preferably, a sample from an animal has been isolated or derived previously from an animal subject by, for example, surgery, or using a syringe or swab.

In another embodiment, a sample can comprise a cell or cell extract or mixture thereof derived from a tissue or organ such as described herein above. Nucleic acid preparation derived from organs, tissues or cells are also particularly useful.

The sample can be prepared on a solid matrix for histological analyses, or alternatively, in a suitable solution such as, for example, an extraction buffer or suspension buffer, and the present disclosure clearly extends to the testing of biological solutions thus prepared. However, in one exemplary embodiment, the high-throughput system of the present disclosure is employed using samples in solution.

In other exemplary embodiments according to the disclosure, an animal thought to have been produced by genetic manipulation can be tested to determine whether a trait exhibited by that animal is due to sexual breeding or whether the trait is present due to genetic manipulation and the animal subsequently cloned, such as by SCNT

Accordingly, the skilled artisan can design probes and/or primers to determine the origin of a phenotypic or genotypic trait. The skilled artisan is aware that a suitable probe or primer i.e., one capable of specifically detecting a marker or foreign allele at a target locus, will specifically hybridize to a region of the genome in genomic DNA from the individual being tested that comprises the marker or allele. As used herein “selectively hybridizes” means that the polynucleotide used as a probe is used under conditions where a target polynucleotide is found to hybridize to the probe at a level significantly above background. The background hybridization may occur because of other polynucleotides present, for example, in genomic DNA being screened. In this event, background implies a level of signal generated by interaction between the probe and non-specific DNA which is less than 10 fold, preferably less than 100 fold as intense as the specific interaction observed with the target DNA. The intensity of interaction are measured, for example, by radiolabelling the probe, e.g. with 32P.

As will be known to the skilled artisan a probe or primer comprises nucleic acid and may consist of synthetic oligonucleotides generally up to about 100-300 nucleotides in length and in some embodiments of about 50-100 nucleotides in length or from about 8-100 or 8-50 nucleotides in length. For example, locked nucleic acid (LNA) or protein-nucleic acid (PNA) probes or molecular beacons for the detection of one or more SNPs are generally at least about 8 to 12 nucleotides in length. Longer nucleic acid fragments up to several kilobases in length can also be used, e.g., derived from genomic DNA that has been sheared or digested with one or more restriction endonucleases. Alternatively, probes/primers can comprise RNA. However, artisans will immediately appreciate that all ranges and values between the explicitly stated bounds are contemplated, with any of the following being available as an upper or lower limit: 10, 50, 100, 120, 130, 150, 200, 250, 300, 350, 400, 450 for example up to at least 1000 nucleotides.

Exemplary probes or primers for use in the present disclosure will be compatible with the high-throughput system described herein. Exemplary probes and primers will comprise locked nucleic acid (LNA) or protein-nucleic acid (PNA) probes or molecular beacons, preferably bound to a solid phase. For example, LNA or PNA probes bound to a solid support are used, wherein the probes each comprise an SNP and sufficient probes are bound to the solid support to span the genome of the species to which an individual being tested belongs.

The number of probes or primers will vary depending upon the number of loci or QTLs being screened and, in the case of genome-wide screens, the size of the genome being screened. The determination of such parameters is readily determined by a skilled artisan without undue experimentation.

Specificity of probes or primers can also depend upon the format of hybridization or amplification reaction employed for genotyping.

The sequence(s) of any particular probe(s) or primer(s) used in the method of the present disclosure will depend upon the locus or QTL or combination thereof being screened. In this respect, the present disclosure can be generally applied to the genotyping of any locus or QTL or to the simultaneous or sequential genotyping of any number of QTLs or loci including genome-wide genotyping. This generality is not to be taken away or read down to a specific locus or QTL or combination thereof. The determination of probe/primer sequences is readily determined by a skilled artisan without undue experimentation

Standard methods are employed for designing probes and/or primers e.g., as described by Dveksler (Eds) (In: PCR Primer: A Laboratory Manual, Cold Spring Harbour Laboratories, N Y, 1995). Software packages are also publicly available for designing optimal probes and/or primers for a variety of assays, e.g., Primer 3 available from the Center for Genome Research, Cambridge, Mass., USA. Probes and/or primers are preferably assessed to determine those that do not form hairpins, self-prime, or form primer dimers (e.g. with another probe or primer used in a detection assay). Furthermore, a probe or primer (or the sequence thereof) is preferably assessed to determine the temperature at which it denatures from a target nucleic acid (i.e. the melting temperature of the probe or primer, or Tm). Methods of determining Tm are known in the art and described, for example, in Santa Lucia, Proc. Natl. Acad. Sci. USA, 95: 1460-1465, 1995 or Bresslauer et al., Proc. Natl. Acad. Sci. USA, 83: 3746-3750, 1986.

For LNA or PNA probes or molecular beacons, in some exemplary embodiments the probe or molecular beacon is at least about 8 to 12 nucleotides in length and more preferably, for the SNP to be positioned at approximately the center of the probe, thereby facilitating selective hybridization and accurate detection.

For detecting one or more SNPs using an allele-specific PCR assay or a ligase chain reaction assay, the probe/primer is generally designed such that the 3′ terminal nucleotide hybridizes to the site of the SNP. The 3′ terminal nucleotide may be complementary to any of the nucleotides known to be present at the site of the SNP. When complementary nucleotides occur in both the probe/primer and at the site of the polymorphism, the 3′ end of the probe or primer hybridizes completely to the marker of interest and facilitates, for example, PCR amplification or ligation to another nucleic acid. Accordingly, a probe or primer that completely hybridizes to the target nucleic acid produces a positive result in an assay.

For primer extension reactions, the probe/primer is generally designed such that it specifically hybridizes to a region adjacent to a specific nucleotide of interest, e.g., an SNP. While the specific hybridization of a probe or primer may be estimated by determining the degree of homology of the probe or primer to any nucleic acid using software, such as, for example, BLAST, the specificity of a probe or primer is generally determined empirically using methods known in the art.

Methods of producing/synthesizing probes and/or primers useful in the present disclosure are known in the art. For example, oligonucleotide synthesis is described, in Gait (Ed) (In: Oligonucleotide Synthesis: A Practical Approach, IRL Press, Oxford, 1984); LNA synthesis is described, for example, in Nielsen et al, J. Chem. Soc. Perkin Trans., 1: 3423, 1997; Singh and Wengel, Chem. Commun. 1247, 1998; and PNA synthesis is described, for example, in Egholm et al., Am. Chem. Soc., 114: 1895, 1992; Egholm et al., Nature, 365: 566, 1993; and Orum et al., Nucl. Acids Res., 21: 5332, 1993.

Numerous methods are known in the art for detecting the occurrence of a particular marker in a sample.

In one exemplary embodiment, a marker is detected using a probe or primer that selectively hybridizes to said marker in a sample from an individual under moderate stringency, and preferably, high stringency conditions. If the probe or primer is detectably labelled with a suitable reporter molecule, e.g., a chemiluminescent label, fluorescent label, radiolabel, enzyme, hapten, or unique oligonucleotide sequence etc., then the hybridization may be detected directly by determining binding of reporter molecule. Alternatively, hybridized probe or primer may be detected by performing an amplification reaction such as polymerase chain reaction (PCR) or similar format, and detecting the amplified nucleic acid. Preferably, the probe or primer is bound to solid support e.g., in the high-throughput system of the present disclosure.

For the purposes of defining the level of stringency to be used in the hybridization, a low stringency is defined herein as hybridization and/or a wash step(s) carried out in 2-6×SSC buffer, 0.1% (w/v) SDS at 28° C., or equivalent conditions. A moderate stringency is defined herein as hybridization and/or a wash step(s) carried out in 0.2-2×SSC buffer, 0.1% (w/v) SDS at a temperature in the range 45° C. to 65° C., or equivalent conditions. A high stringency is defined herein as hybridization and/or a wash step(s) carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample DNA, or the type of hybridization probe used.

Progressively higher stringency conditions can also be employed wherein the stringency is increased stepwise from lower to higher stringency conditions. Exemplary progressive stringency conditions are as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

For example, the modification of a sequence of a region (haplotype) of the genome or an expression product thereof, such as, for example, an insertion (e.g., introduction of a foreign allele at a target locus), a deletion, a transversion or a transition, is detected using a method, such as, polymerase chain reaction (PCR), strand displacement amplification, ligase chain reaction, cycling probe technology or a DNA microarray chip amongst others.

Methods of PCR are known in the art and described, for example, in Dieffenbach (ed.) and Dveksler (ed) (In: PCR Primer: A Laboratory Manual, Cold Spring Harbour Laboratories, N Y, 1995). Generally, for PCR two non-complementary nucleic acid primer molecules comprising at least about 15 nucleotides, more preferably at least 20 nucleotides in length are hybridized to different strands of a nucleic acid template molecule, and specific nucleic acid molecule copies of the template are amplified enzymatically. PCR products may be detected using electrophoresis and detection with a detectable marker that binds nucleic acids. Alternatively, one or more of the oligonucleotides is/are labeled with a detectable marker (e.g. a fluorophore) and the amplification product detected using, for example, a lightcycler (Perkin Elmer, Wellesley, Mass., USA). Clearly, the present disclosure also encompasses quantitative forms of PCR, such as, for example, Taqman assays.

Strand displacement amplification (SDA) utilizes oligonucleotides, a DNA polymerase and a restriction endonuclease to amplify a target sequence. The oligonucleotides are hybridized to a target nucleic acid and the polymerase used to produce a copy of this region. The duplexes of copied nucleic acid and target nucleic acid are then nicked with an endonuclease that specifically recognizes a sequence at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

Ligase chain reaction (described, for example, in EP 320,308 and U.S. Pat. No. 4,883,750) uses at least two oligonucleotides that bind to a target nucleic acid in such a way that they are adjacent. A ligase enzyme is then used to link the oligonucleotides. Using thermocycling the ligated oligonucleotides then become a target for further oligonucleotides. The ligated fragments are then detected, for example, using electrophoresis, or MALDI-TOF. Alternatively, or in addition, one or more of the probes is labeled with a detectable marker, thereby facilitating rapid detection.

Cycling Probe Technology uses chimeric synthetic probe that comprises DNA-RNA-DNA that is capable of hybridizing to a target sequence. Upon hybridization to a target sequence the RNA-DNA duplex formed is a target for RNase H thereby cleaving the probe. The cleaved probe is then detected using, for example, electrophoresis or MALDI-TOF.

Additional methods for detecting SNPs are known in the art, and reviewed, for example, in Landegren et al, Genome Research 8: 769-776, 1998) (hereby incorporated by reference in its entirety).

For example, an SNP that introduces or alters a sequence that is a recognition sequence for a restriction endonuclease is detected by digesting DNA with the endonuclease and detecting the fragment of interest using, for example, Southern blotting (described in Ausubel et al (In: Current Protocols in Molecular Biology. Wiley Interscience, ISBN 047 150338, 1987) (herein incorporated by reference in its entirety) and Sambrook et al (In: Molecular Cloning: Molecular Cloning: A Laboratory Manual, Cold Spring Harbor Laboratories, New York, Third Edition 2001) (herein incorporated by reference in its entirety). Alternatively, a nucleic acid amplification method described supra, is used to amplify the region surrounding the SNP. The amplification product is then incubated with the endonuclease and any resulting fragments detected, for example, by electrophoresis, MALDI-TOF or PCR.

The direct analysis of the sequence of polymorphisms of the present disclosure can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988) (incorporated herein by reference in its entirety). For example, a region of genomic DNA comprising one or more markers is amplified using an amplification reaction, e.g., PCR, and following purification of the amplification product, the amplified nucleic acid is used in a sequencing reaction to determine the sequence of one or both alleles at the site of an SNP of interest.

Alternatively, one or more SNPs is/are detected using single stranded conformational polymorphism (SSCP). SSCP relies upon the formation of secondary structures in nucleic acids and the sequence dependent nature of these secondary structures. In one form of this analysis, an amplification method, such as, for example, a method described supra, is used to amplify a nucleic acid that comprises an SNP. The amplified nucleic acids are then denatured, cooled and analyzed using, for example, non-denaturing polyacrylamide gel electrophoresis, mass spectrometry, or liquid chromatography (e.g., HPLC or dHPLC). Regions that comprise different sequences form different secondary structures, and as a consequence migrate at different rates through, for example, a gel and/or a charged field. Clearly, a detectable marker may be incorporated into a probe/primer useful in SSCP analysis to facilitate rapid marker detection.

Alternatively, any nucleotide changes may be detected using, for example, mass spectrometry or capillary electrophoresis. For example, amplified products of a region of DNA comprising an SNP from a test sample are mixed with amplified products from an individual having a known genotype at the site of the SNP. The products are denatured and allowed to re-anneal. Those samples that comprise a different nucleotide at the position of the SNP will not completely anneal to a nucleic acid molecule from the control sample thereby changing the charge and/or conformation of the nucleic acid, when compared to a completely annealed nucleic acid. Such incorrect base pairing is detectable using, for example, mass spectrometry.

Allele-specific PCR (as described, for example, In Liu et al, Genome Research, 7:389-398, 1997) (herein incorporated by reference in its entirety) is also useful for determining the presence of one or other allele of an SNP. An oligonucleotide is designed, in which the most 3′ base of the oligonucleotide hybridizes to a specific form of an SNP of interest (i.e., allele). During a PCR reaction, the 3′ end of the oligonucleotide does not hybridize to a target sequence that does not comprise the particular form of the SNP detected. Accordingly, little or no PCR product is produced, indicating that a base other than that present in the oligonucleotide is present at the site of SNP in the sample. PCR products are then detected using, for example, gel or capillary electrophoresis or mass spectrometry.

Primer extension methods (described, for example, in Dieffenbach (ed.) and Dveksler (ed) (In: PCR Primer: A Laboratory Manual, Cold Spring Harbour Laboratories, N Y, 1995)) are also useful for the detection of an SNP. An oligonucleotide is used that hybridizes to the region of a nucleic acid adjacent to the SNP. This oligonucleotide is used in a primer extension protocol with a polymerase and a free nucleotide diphosphate that corresponds to either or any of the possible bases that occur at the site of the SNP. Preferably, the nucleotide-diphosphate is labeled with a detectable marker (e.g. a fluorophore). Following primer extension, unbound labeled nucleotide diphosphates are removed, e.g. using size exclusion chromatography or electrophoresis, or hydrolyzed, using for example, alkaline phosphatase, and the incorporation of the labeled nucleotide into the oligonucleotide is detected, indicating the base that is present at the site of the SNP. Alternatively, or in addition, as exemplified herein primer extension products are detected using mass spectrometry (e.g., MALDI-TOF).

The present disclosure extends to high-throughput forms of primer extension analysis, such as, for example, minisequencing (Sy Vamen et al., Genomics 9: 341-342, 1995) (incorporated by reference in its entirety) wherein a probe or primer or multiple probes or primers is/are immobilized on a solid support (e.g. a glass slide), a sample comprising nucleic acid is brought into contact with the probe(s) or primer(s), a primer extension reaction is performed wherein each of the free nucleotide bases A, C, G, T is labeled with a different detectable marker and the presence or absence of one or more SNPs is determined by determining the detectable marker bound to each probe and/or primer.

Fluorescently labeled locked nucleic acid (LNA) molecules or fluorescently labeled protein-nucleic acid (PNA) molecules are useful for the detection of SNPs (as described in Simeonov and Nikiforov, Nucleic Acids Research, 30(17): 1-5, 2002). LNA and PNA molecules bind, with high affinity, to nucleic acid, in particular, DNA. Flurophores (in particular, rhodomine or hexachlorofluorescein) conjugated to the LNA or PNA probe fluoresce at a significantly greater level upon hybridization of the probe to target nucleic acid compared to a probe that has not hybridized to a target nucleic acid. However, the level of increase of fluorescence is not enhanced to the same level when even a single nucleotide mismatch occurs. Accordingly, the degree of fluorescence detected in a sample is indicative of the presence of a mismatch between the LNA or PNA probe and the target nucleic acid, such as, in the presence of an SNP. Preferably, fluorescently labeled LNA or PNA technology is used to detect a single base change in a nucleic acid that has been previously amplified using, for example, an amplification method described supra.

As will be apparent to the skilled artisan, LNA or PNA detection technology is amenable to a high-throughput detection of one or more markers immobilizing an LNA or PNA probe to a solid support, as described in Drum et al., Clin. Chem, 45: 1898-1905, 1999 (incorporated herein in its entirety).

Similarly, Molecular Beacons are useful for detecting SNPs directly in a sample or in an amplified product (see, for example, Mhlang and Malmberg, Methods 25: 463-471, 2001) (incorporated herein in its entirety). Molecular beacons are single stranded nucleic acid molecules with a stem-and-loop structure. The loop structure is complementary to the region surrounding the SNP of interest. The stem structure is formed by annealing two “arms” complementary to each other on either side of the probe (loop). A fluorescent moiety is bound to one arm and a quenching moiety that suppresses any detectable fluorescence when the molecular beacon is not bound to a target sequence bound to the other arm. Upon binding of the loop region to its target nucleic acid the arms are separated and fluorescence is detectable. However, even a single base mismatch significantly alters the level of fluorescence detected in a sample. Accordingly, the presence or absence of a particular base at the site of an SNP is determined by the level of fluorescence detected.

The present disclosure also encompasses other methods of detecting a genetic marker such as a unique sequence, SNP or foreign allele, such as, for example, microarrays (commercially available from, for example, Affymetrix, or described, for example, in U.S. Pat. No. 6,468,743 (incorporated herein in its entirety) or Hacia et al, Nature Genetics, 14: 441, 1996) (incorporated herein in its entirety), Taqman® Assays (commercially available from, for example, LifeTechnologies and described in Livak et al, Nature Genetics, 9:341-342, 1995) (incorporated herein by reference in its entirety), solid phase minisequencing (as described in Syvamen et al. Genomics, 13: 1008-1017, 1992) (incorporated herein by reference in its entirety), minisequencing with FRET (as described in Chen and Kwok, Nucleic Acids Res. 25: 347-353, 1997) (incorporated herein by reference in its entirety) or pyrominisequencing (as reviewed in Landegren et al., Genome Res., 8(8): 769-776, 1998) (incorporated herein by reference in its entirety).

In those cases in which the polymorphism or marker occurs in a region of nucleic acid that encodes RNA, said polymorphism or marker is detected using a method such as, for example, RT-PCR, NASBA or TMA (transcription mediated amplification).

Methods of RT-PCR are known in the art and described, for example, in Dieffenbach (ed.) and Dveksler (ed) (In: PCR Primer: A Laboratory Manual, Cold Spring Harbour Laboratories, N Y, 1995 (incorporated herein by reference in its entirety).

Methods of TMA or self-sustained sequence replication (3SR) use two or more oligonucleotides that flank a target sequence, an RNA polymerase, RNase H and a reverse transcriptase. One oligonucleotide (that also comprises a RNA polymerase binding site) hybridizes to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce an RNA copy of the cDNA, and the process repeated.

NASBA systems relies on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridizes to the target sequence and comprises a RNA polymerase binding site at its 5′ end. The template RNA is digested with RNase H and double stranded DNA is synthesized. The RNA polymerase then produces multiple RNA copies of the cDNA and the process is repeated.

The hybridization to and/or amplification of a marker is detectable using, for example, electrophoresis and/or mass spectrometry. In this regard, one or more of the probes/primers and/or one or more of the nucleotides used in an amplification reactions may be labeled with a detectable marker to facilitate rapid detection of a marker, for example, a fluorescent label (e.g. Cy5 or Cy3) or a radioisotope (e.g. 32P).

Alternatively, amplification of a nucleic acid may be continuously monitored using a melting curve analysis method, such as that described in, for example, U.S. Pat. No. 6,174,670. Such methods are suited to determining the level of an alternative splice form in a biological sample.

Methods of the disclosure can identify nucleotide occurrences at SNPs using genome-wide sequencing or “microsequencing” methods. Whole-genome sequencing of individuals identifies all SNP genotypes in a single analysis. Microsequencing methods determine the identity of only a single nucleotide at a “predetermined” site. Such methods have particular utility in determining the presence and identity of polymorphisms in a target polynucleotide. Such microsequencing methods, as well as other methods for determining the nucleotide occurrence at SNP loci are discussed in Boyce-Jacino, et al., U.S. Pat. No. 6,294,336, incorporated herein by reference.

Microsequencing methods include the Genetic Bit Analysis method disclosed by Goelet, P. et al. (WO 92/15712, herein incorporated by reference). Additional, primer-guided, nucleotide incorporation procedures for assaying polymorphic sites in DNA have also been described (Komher et al, Nucl. Acids. Res. 17, 7779-7784, 1989; Sokolov, Nucl. Acids Res. 18, 3671 (1990); Syvanen et al., Genomics 8, 684-692, 1990; Kuppuswamy et al., Proc. Natl. Acad. Sci. (U.S.A.) 88, 1143-1147, 1991; Prezant et al, Hum. Mutat. 1, 159-164, 1992; Ugozzoli et al., GATA 9, 107-112, 1992; Nyren et al., Anal. Biochem. 208, 171-175, 1993; Wallace, WO 89/10414; Mundy, U.S. Pat. No. 4,656,127; Cohen et al., French Pat. No. 2,650,840; WO 91/02087). In response to the difficulties encountered in employing gel electrophoresis to analyze sequences, alternative methods for microsequencing have been developed, e.g., Macevicz, U.S. Pat. No. 5,002,867 incorporated herein by reference. Boyce-Jacino et al., U.S. Pat. No. 6,294,336 provide a solid phase sequencing method for determining the sequence of nucleic acid molecules (either DNA or RNA) by utilizing a primer that selectively binds a polynucleotide target at a site wherein the SNP is the most 3′ nucleotide selectively bound to the target. Oliphant et al., Suppl. Biotechniques, June 2002, describe the use of BeadArray™ Technology to determine the nucleotide occurrence of an SNP, Alternatively, nucleotide occurrences for SNPs can be determined using a DNA MassArray system (Sequenom, Inc., San Diego, Calif.) is used, which system combines SpectroChips™, microfluidics, nanodispensing, biochemistry, and MALDI-TOF MS (matrix-assisted laser desorption ionization time of flight mass spectrometry).

Particularly useful methods include those that are readily adaptable to a high throughput format, to a multiplex format, or to both. High-throughput systems for analyzing markers, especially SNPs, can include, for example, a platform such as the UHT SNP-IT™ platform (Orchid Biosciences, Princeton, N.J., USA) MassArray™ system (Sequenom, Inc., San Diego, Calif., USA), the integrated SNP genotyping system (Illumina, San Diego, Calif., USA), TaqMan™ (ABI, Foster City, Calif., USA), Rolling circle amplification, fluorescent polarization, amongst others described herein above. In general, SNP-IT™ is a 3-step primer extension reaction. In the first step, a target polynucleotide is isolated from a sample by hybridization to a capture primer, which provides a first level of specificity. In a second step the capture primer is extended from a terminating nucleotide trisphosphate at the target SNP site, which provides a second level of specificity. In a third step, the extended nucleotide trisphosphate can be detected using a variety of known formats, including: direct fluorescence, indirect fluorescence, an indirect colorimetric assay, mass spectrometry, fluorescence polarization, etc. Reactions can be processed in 384 well format in an automated format using an SNPstream™ instrument (Orchid BioSciences, Princeton, N.J.).

### High Throughput System for Genotypic Selection

The instant disclosure also provides a high-throughput system for genotypic selection in a population having a small effective population size, in some embodiments, the system comprises a solid support consisting essentially of or having nucleic acids of different sequence bound directly or indirectly thereto, wherein each nucleic acid of different sequence comprises a polymorphic genetic marker derived from an ancestor or founder that is representative of the current population.

Exemplary high-throughput systems are hybridization mediums e.g., a microfluidic device or homogenous assay medium. Numerous microfluidic devices are known that include solid supports with microchannels (See e.g., U.S. Pat. Nos. 5,304,487, 5,110,745, 5,681,484, and 5,593,838). In one exemplary embodiment, the high throughput system comprises an SNP chip comprising 10,000-100,000 oligonucleotides each of which consists of a sequence comprising an SNP. Each of these hybridization mediums is suitable for determining the presence or absence of a marker associated with a trait.

The nucleic acids are typically oligonucleotides, attached directly or indirectly to the solid support. Accordingly, the oligonucleotides are used to determine the nucleotide occurrence of a marker associated with a trait, by virtue of the hybridization of nucleic acid from the subject being tested to an oligonucleotide of a series of oligonucleotides bound to the solid support being affected by the nucleotide occurrence of the marker in question e.g., by the presence or absence of an SNP in the subject's nucleic acid. Accordingly, oligonucleotides can be selected that bind at or near a genomic location of each marker. Such oligonucleotides can include forward and reverse oligonucleotides that can support amplification of a particular polymorphic marker present in template nucleic acid obtained from the subject being tested. Alternatively, or in addition, the oligonucleotides can include extension primer sequences that hybridize in proximity to a marker to thereby support extension to the marker for the purposes of identification. A suitable detection method will detect binding or tagging of the oligonucleotides e.g., in a genotyping method described herein.

Techniques for producing immobilized arrays of DNA molecules have been described in the art. Generally, most methods describe how to synthesize single-stranded nucleic acid molecule arrays, using for example masking techniques to build up various permutations of sequences at the various discrete positions on the solid substrate. U.S. Pat. No. 5,837,832 (hereby incorporated by reference in its entirety), the contents of which are incorporated herein by reference, describes an improved method for producing DNA arrays immobilized to silicon substrates based on very large scale integration technology. In particular, U.S. Pat. No. 5,837,832 (hereby incorporated by reference in its entirety) describes a strategy called “tiling” to synthesize specific sets of probes at spatially-defined locations on a substrate which are used to produce the immobilized DNA array. U.S. Pat. No. 5,837,832 (hereby incorporated by reference in its entirety) also provides references for earlier techniques that may also be used.

DNA can be synthesized in situ on the surface of the substrate. However, DNA may also be printed directly onto the substrate using for example robotic devices equipped with either pins or piezo electric devices. Microarrays are generally produced step-wise, by the in situ synthesis of the target directly onto the support, or alternatively, by exogenous deposition of pre-prepared targets. Photolithography, mechanical microspotting, and ink jet technology are generally employed for producing microarrays.

In photolithography, a glass wafer, modified with photolabile protecting groups, is selectively activated e.g., for DNA synthesis, by shining light through a photomask. Repeated deprotection and coupling cycles enable the preparation of high-density oligonucleotide microarrays (see for example, U.S. Pat. No. 5,744,305, issued Apr. 28, 1998) (hereby incorporated by reference in its entirety).

Microspotting encompasses deposition technologies that enable automated microarray production, by printing small quantities of pre-made target substances onto solid surfaces. Printing is accomplished by direct surface contact between the printing substrate and a delivery mechanism, such as a pin or a capillary. Robotic control systems and multiplexed print heads allow automated microarray fabrication.

Ink jet technologies utilize piezoelectric and other forms of propulsion to transfer biochemical substances from miniature nozzles to solid surfaces. Using piezoelectricity, the target sample is expelled by passing an electric current through a piezoelectric crystal which expands to expel the sample. Piezoelectric propulsion technologies include continuous and drop-on-demand devices. In addition to piezoelectric ink jets, heat may be used to form and propel drops of fluid using bubble-jet or thermal ink jet heads; however, such thermal ink jets are typically not suitable for the transfer of biological materials due to the heat which is often stressful on biological samples. Examples of the use of ink jet technology include U.S. Pat. No. 5,658,802 (hereby incorporated by reference in its entirety).

A plurality of nucleic acids is typically immobilized onto or in discrete regions of a solid substrate. The substrate is porous to allow immobilization within the substrate, or substantially non-porous to permit surface immobilization.

The solid substrate can be made of any material to which polypeptides can bind, either directly or indirectly. Examples of suitable solid substrates include flat glass, silicon wafers, mica, ceramics and organic polymers such as plastics, including polystyrene and polymethacrylate. It is also possible to use semi-permeable membranes such as nitrocellulose or nylon membranes, which are widely available. The semi-permeable membranes are mounted on a more robust solid surface such as glass. The surfaces may optionally be coated with a layer of metal, such as gold, platinum or other transition metal.

Preferably, the solid substrate is generally a material having a rigid or semi-rigid surface. In some embodiments, at least one surface of the substrate will be substantially flat, although in some embodiments it are desirable to physically separate synthesis regions for different polymers with, for example, raised regions or etched trenches. It is also an embodiment that the solid substrate is suitable for the high density application of DNA sequences in discrete areas of typically from 50 to 100 μm, giving a density of 10,000 to 40,000 cm-2.

The solid substrate is conveniently divided up into sections. This is achieved by techniques such as photoetching, or by the application of hydrophobic inks, for example Teflon-based inks (Cel-line, USA).

Discrete positions, in which each different member of the array is located may have any convenient shape, e.g., circular, rectangular, elliptical, wedge-shaped, etc.

Attachment of the nucleic acids to the substrate can be covalent or non-covalent, generally via a layer of molecules to which the nucleic acids bind. For example, the nucleic acid probes/primers can be labelled with biotin and the substrate coated with avidin and/or streptavidin. A convenient feature of using biotinylated probes/primers is that the efficiency of coupling to the solid substrate is determined easily.

A chemical interface may be provided between the solid substrate e.g., in the case of glass, and the probes/primers. Examples of suitable chemical interfaces include hexaethylene glycol, polylysine. For example, polylysine can be chemically modified using standard procedures to introduce an affinity ligand.

Other methods for attaching the probes/primers to the surface of a solid substrate include the use of coupling agents known in the art, e.g., as described in WO 98/49557 (hereby incorporated by reference in its entirety).

The high-throughput system of the present disclosure is designed to determine nucleotide occurrences of one SNP or a series of SNPs. The systems can determine nucleotide occurrences of an entire genome-wide high-density SNP map.

High-throughput systems for analyzing markers, especially SNPs, can include, for example, a platform such as the UHT SNP-IT platform (Orchid Biosciences, Princeton, N.J., USA) MassArray™ system (Sequenom, San Diego, Calif., USA), the integrated SNP genotyping system (Illumina, San Diego, Calif., USA), TaqMan™ (ABI, Foster City, Calif., USA). Exemplary nucleic acid arrays are of the type described in WO 95/11995 (hereby incorporated by reference in its entirety), WO 95/11995 (hereby incorporated by reference in its entirety) also describes sub-arrays optimized for detection of a variant form of a pre-characterized polymorphism. Such a sub-array contains probes designed to be complementary to a second reference sequence, which is an allelic variant of the first reference sequence. The inclusion of a second group (or further groups) can be particularly useful for analyzing short sub-sequences of a primary reference sequence in which multiple mutations are expected to occur within a short distance commensurate with the length of the probes (e.g., two or more mutations within 9 to 21 bases). More preferably, the high throughput system comprises a SNP microarray such as those available from Affymetrix or described, for example, in U.S. Pat. No. 6,468,743 (hereby incorporated by reference in its entirety) or Hacia et al, Nature Genetics, 14:441, 1996 (hereby incorporated by reference in its entirety).

DNA arrays are typically read at the same time by charged coupled device (CCD) camera or confocal imaging system. Alternatively, the DNA array can be placed for detection in a suitable apparatus that can move in an x-y direction, such as a plate reader. In this way, the change in characteristics for each discrete position are measured automatically by computer controlled movement of the array to place each discrete element in turn in line with the detection means.

The detection means is capable of interrogating each position in the library array optically or electrically. Examples of suitable detection means include CCD cameras or confocal imaging systems.

The system can further include a detection mechanism for detecting binding the series of oligonucleotides to the series of SNPs. Such detection mechanisms are known in the art.

The high-throughput system of the present disclosure can include a reagent handling mechanism that can be used to apply a reagent, typically a liquid, to the solid support.

The high-throughput system can also include a mechanism effective for moving a solid support and a detection mechanism.

Recently methods have been identified to make genetically modified animals using custom nucleases (TALENs) to afford efficient nonmeiotic introgression of foreign alleles into a haplotype. Such modifications can be made by identifying the specific sequence structure of a gene or allele and the sequence or sequences surrounding it on its chromosome. Due to rapid advances in molecular biology, gene sequencing and animal cloning, the ability to identify discrete genes and identify or hypothesize their function due to homology with known genes of other species has provided scientists an ability, not only to attempt to modify an animals native genetic material but to insert homologous genes (i.e., exogenous genes) from other animals, e.g., different breeds, lineages, strains or species, into an identified locus which may be the site of a native gene or allele of a host species. Further, the benefit of such insertion or introgression, is that a specific trait can be conferred on a species or breed in a single generation instead of via traditional methods of livestock breeding, cross breeding and backcrossing to confer a trait without adding of unwanted genes from the donor on the host animal. For example, U.S. Pub No. 2012/0222143, hereby incorporated in its entirety for all purposes, describes methods of using non-meiotic introgression to introduce desirable traits into livestock genome to produce a stably expressed phenotype.

Using precision gene editing, a target allele in an animal's genome and more particular a target allele within a host animals recognized haplotype can be edited with an insertion or deletion as desired. For example, efficient nonmeiotic introgression can be used to change a single base or it can be used to insert simple alleles or complex alleles, in phase, to express new traits without altering other genes, alleles or phenotypic traits that are particular to any breed of livestock.

In such examples, it may become necessary to identify whether a particular livestock animal exhibits (or lacks) some trait that is foreign to its recognized phenotype and whether the presence of that trait (or lack thereof) is the result of precise gene editing or is the result of a random mutation or sexual cross breeding with another animal that confers such trait. In such cases, the presence of a foreign or exogenous allele within a known haplotype of a domesticated animal can be identified. Those of skill in the art will appreciate that if known DNA sequences (or markers) contained within a haplotype can be identified and if foreign DNA were introduced into that haplotype at a target locus, it would be possible to identify the animal due to the presence of the inserted DNA at the site of a native allele (e.g., the target locus) within the haplotype.

Alleles on a chromosome that are in close proximity to each other exhibit a marked linkage disequilibrium such that the alleles do not segregate independently. Rather, alleles within 500 bases of each other have a high probability of segregating together even when an animal exhibiting those alleles is the product of sexual reproduction. Further, those of skill in the art recognize that the closer alleles are on a chromosome the greater the chance that they will co-segregate. Therefore, alleles and/or SNPs closer to the target locus than 500 bases can also be used as haplotype markers in some cases alleles within 200 bases, 100 bases or 50 bases will be identified. Advances in technology have provided the ability to sequence whole genomes of animals.

For example, the National Animal Genome Research Program which has a goal of coordinating the genomic sequencing of livestock including, cattle, pigs, chickens, sheep, horses and organisms used in aquaculture (See, for example, http://www.animalgenome.org/). In some cases, because livestock have been the subject of centuries of breeding and cross breeding to obtain desirable breeds, the genetic diversity of such breeds is limited. For example, Hayes et al., (U.S. Pub. No. 2014/0220575) (hereby incorporated by reference in its entirety) estimate that the effective population size (Ne) of popular livestock breeds is extremely small when compared to their large numbers and geographic distribution. For example, among cattle, the Ne of Holstein-Friesians is estimated to be between 50 and 100; Brown Swiss about 46; Holstein 49 and Danish Red 47. In sheep, the Ne for Dorset-Rarnboulliet-Finnsheep cross is 35. Pigs have a slightly high Ne estimated at <200 for Harmegnies; 85 for Duroc/Large white and 300 for Large white. Chicken show similarly small Ne for breeds such as Layers which are estimated to be between 91 and 123.

Consequently, from a breeding standpoint and from an economics standpoint, it is important to identify the source of a phenotypic trait foreign to an animal whether that arises from a natural mutation, from sexual reproduction, or whether the trait arises from a foreign allele introduced into a cloned animal. As discussed above, if a trait, such as POLLED, is introduced into a horned breed of cattle via crossing and backcrossing from, for example, a horned breed (Holstein) and a polled breed (angus) and followed by backcrossing the polled progeny with Holstein to arrive at a conventionally acceptable (e.g., subjectively phenotypic) POLLED Holstein, besides taking many generations, the genotypic results of that cross will include portions of the polled genome that are in linkage disequilibrium with the polled allele (whether expressed or not). Introducing such portions of the Angus genome may not be beneficial to the Holstein breed and may alter other traits or characteristics as well. Further, the presence of Angus alleles linked to the polled allele in the Holstein haplotype would be definitive of the introduction of the polled gene by sexual crossing. Conversely, if the polled gene was present in the haplotype due to being introduced into the haplotype by genetic manipulation, (i.e., non-meiotic introgression) the native haplotype of the host breed (Holstein) remains the same with the exception of the introduced Angus allele, nucleotide or nucleotides.

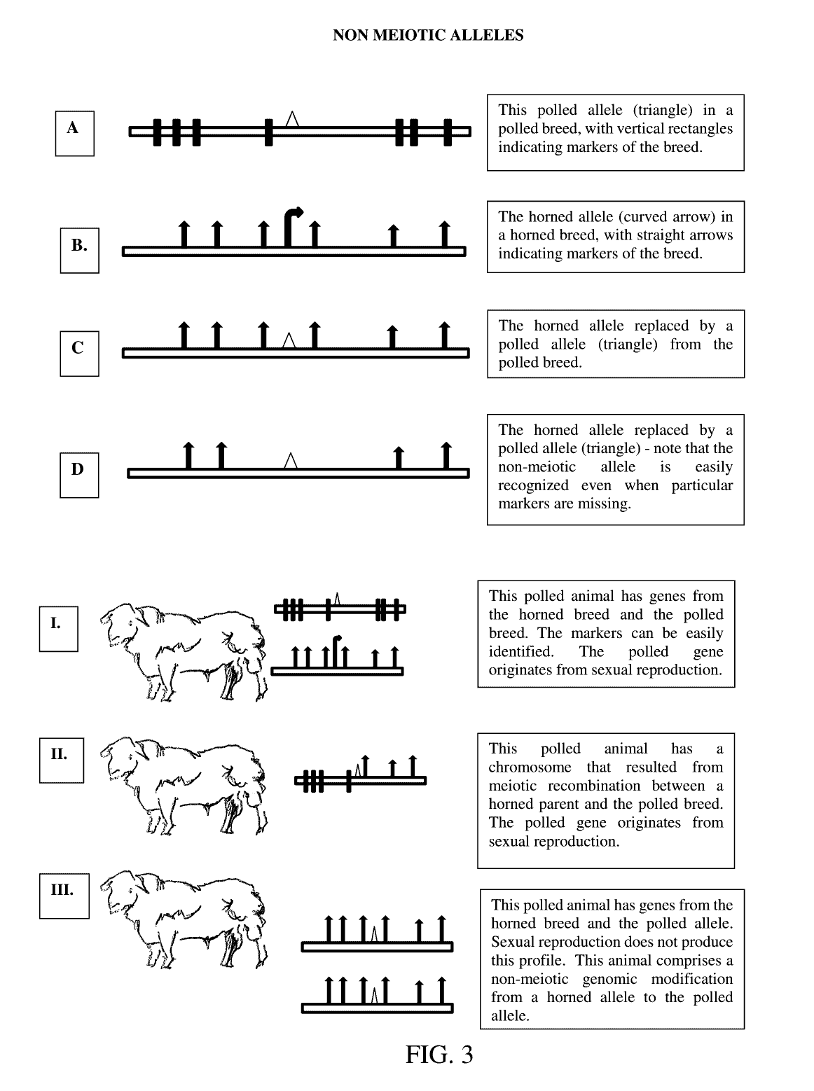

In this respect, while the use of specific gene editing, such as for example, using TALENs to introduce homology directed repair (HDR) requires a knowledge of the native nucleotide sequence being replaced or interrupted, upstream or downstream sequences are irrelevant. However, as recognized by the present inventors, by identifying markers native to the host haplotype along with the correct insertion site for introduced (exogenous) DNA, it is possible to determine whether a foreign allele has been introduced into the host animal molecularly or whether a non-native allele has appeared in an animal by random mutation or by sexual reproduction. See, FIG. 3. This knowledge provides the owner of the animal, the breeder of the animal and the consumer of such animals the comfort of knowing that, except for the introduced, exogenous allele, the animal remains genetically the same as its host/parent.

Accordingly, in one exemplary embodiment, the disclosure comprises a method to identify whether a host animal expressing a foreign allele or phenotype the result of a spontaneous mutation, selective is breeding or is the result of genetic modification. Therefore, in this embodiment, the method includes identifying the presence of a known exogenous allele at a specific, defined locus within a haplotype. The method further includes identifying two or more markers native to the haplotype. In some exemplary embodiments, the markers flank the target locus on a chromosome. In other embodiments, the markers are on the same side of a target locus on a chromosome. Of course, those of skill in the art will appreciate that there may be more than two haplotype markers. For example, there may be three, four or five haplotype markers. The markers may flank the target locus on a chromosome or the markers may be all on the same side of the target locus on a chromosome.

Various exemplary embodiments of devices and compounds as generally described above and methods according to this disclosure, will be understood more readily by reference to the following examples, which are provided by way of illustration and are not intended to be limiting of the disclosure in any fashion.

### Example 1

### Efficient Nonmeiotic Allele Introgression in Livestock

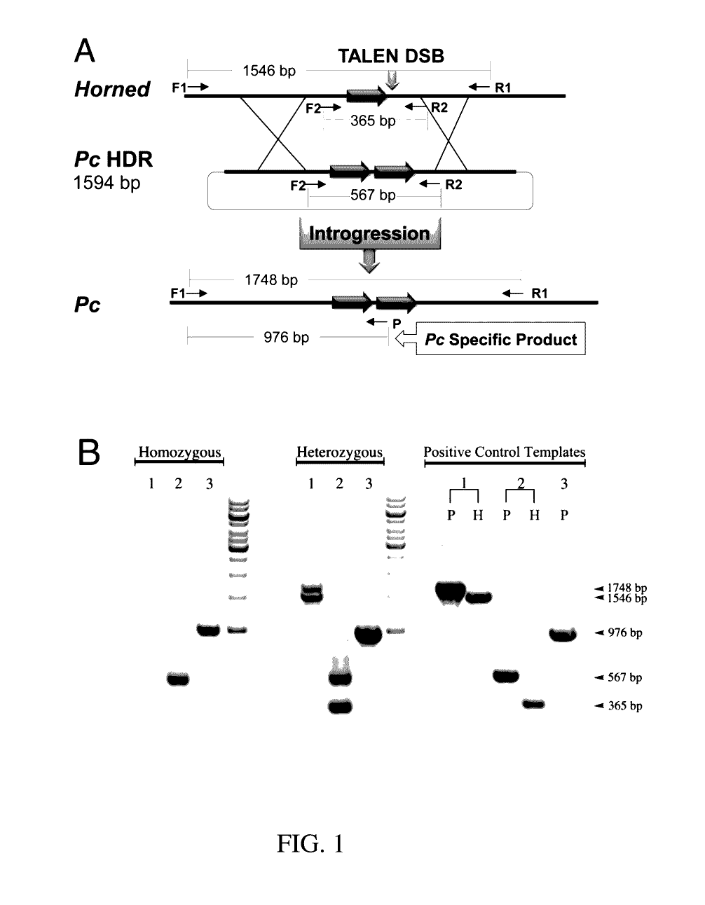

Some beef breeds are naturally horn-free (e.g., Angus), a dominant trait referred to as POLLED (27). Two allelic variants conferring polledness have been identified on chromosome 1 (28). Meiotic introgression of the POLLED trait into horned dairy breeds can be accomplished by traditional crossbreeding, but the genetic merit of the resulting animals would rank lower owing to the admixture of unselected (inferior) alleles for net merit (i.e., milk production) into the population. The inventors undertook the nonmeiotic introgression of the Celtic POLLED allele (duplication of 212 bp that replaces 10 bp), referred to as Pc, into fibroblasts derived from horned dairy bulls. A plasmid HDR template was constructed containing a 1,594-bp fragment including the Pc allele from the Angus breed (FIG. 1A). TALENs were designed such that they could cleave the HORNED allele but leave the Pc allele unaffected. In addition, after finding that one pair of TALENs delivered as mRNA had similar activity as plasmid DNA (Tan et al., Proc Natl Acad Sci USA. 2013 Oct. 8; 110(41):16526-31.), we chose to deliver TALENs as mRNA to eliminate the possible genomic integration of TALEN expression plasmids. Five of 226 colonies (2%) passed each PCR test shown to confirm introgression of Pc (FIG. 1B).

Pc HDR template. A 1,784 bp fragment encompassing the Celtic POLLED allele was PCR amplified (F1: 5′-GGGCAAGTTGCTCAGCTGTTTTTG (SEQ ID 1); R1-5′-TCCGCATGGTTTAGCAGGATTCA (SEQ ID 2)) from Angus genomic DNA and TOPO cloned into the PCR 2.1 vector (Life Technologies). This plasmid was used as positive the control template for analytical primer sets and for derivation of the 1,592 bp HDR template by PCR with following primers (1594 F1: 5′-ATCGAACCTGGGTCTTCTGCATTG (SEQ ID 3); R1: 5′-TCCGCATGGTTTAGCAGGATTCA (SEQ ID 4)) and TOPO cloned as before. Each plasmid was sequence verified prior to use. Transfection grade plasmid was prepared using the Fast-Ion MIDI Plasmid Endo-Free kit (IBI Scientific) and 5 μg or 10 μg was transfected along with 2 μg HP1.3 TALEN mRNA.

### Example 2

### Tissue Culture and Transfection

The bovine POLLED allele was introgressed into a horned bull fibroblast. Cattle fibroblasts were maintained at 30° C. Each transfection was comprised of 500,000-600,000 cells resuspended in buffer “R” mixed with mRNA and oligos and electroporated using the 100 ul tips by the following parameters: input voltage; 1800V; Pulse Width; 20 ms; and pulse number; 1. Typically, 2-4 μg of TALEN expression plasmid or 1-2 μg of TALEN mRNA and 2-3 μM of oligos specific for the gene of interest were included in each transfection. Deviation from those amounts is indicated in the figure legends for both TALENs and CRISPR/Cas9 experiments. After transfection, cells were divided 60:40 into two separate wells of a 6-well dish for three days' culture at either 30° C. or 37° C. respectively. After three days, cell populations were expanded and at 37° C. until at least day 10 to assess stability of edits.

Three days post transfection, 50 to 250 cells were seeded onto 10 cm dishes and cultured until individual colonies reached circa 5 mm in diameter. At this point, 6 ml of TrypLE (Life Technologies) 1:5 (vol/vol) diluted in PBS was added and colonies were aspirated, transferred into wells of a 24-well dish well and cultured under the same conditions. Colonies reaching confluence were collected and divided for cryopreservation and genotyping.

### Example 3

### Confirmation of Introgression

Three of the five clones were homozygous for Pc introgression and were confirmed by sequencing, as is known in the art. Briefly, detection of Pc introgression was performed by PCR using the F1 primer (see above) and the “P” primer (5′-ACGTACTCTTCATTTCACAGCCTAC) (SEQ ID 5) using 1× MyTaq Red mix (Bioline) for 38 cycles (95° C., 25 s; 62° C., 25 s; 72° C., 60 s). A second PCR assay was performed using (F2: 5′-GTCTGGGGTGAGATAGTTTTCTTGG (SEQ ID 6); R2-5′-GGCAGAGATGTTGGTCTTGGGTGT (SEQ ID 7)). Candidates passing both tests were analyzed by PCR using the flanking F1 and R1 primers followed by TOPO cloning and sequencing.

### Example 4

### Identification of Haplotype Markers Confirming Allele Introgression

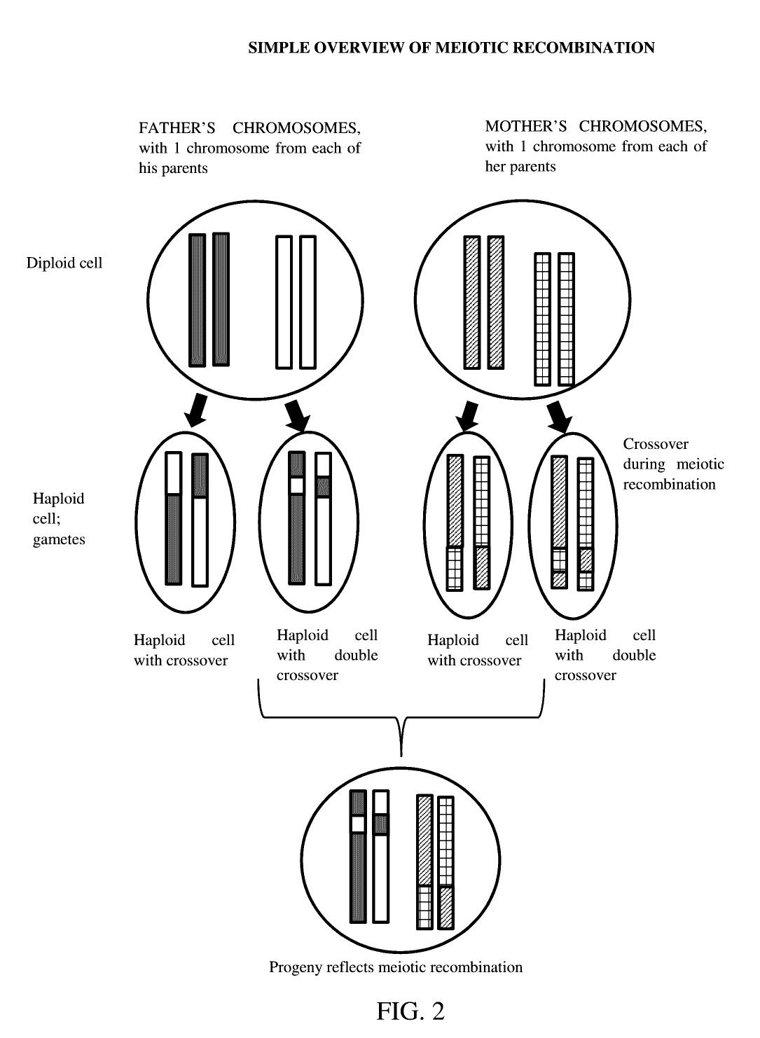

Since the location of the insertion locus for the exogenous DNA is defined, markers for detecting the presence of the haplotype can be designed. As discussed above, a genetic marker may be any known DNA sequence within a chromosome segment. Because of linkage disequilibrium and non-independent assortment, alleles within a haplotype segregate together during crossing over due to various factors but including distance from the target locus. (FIG. 2). Identification of appropriate markers is made by identifying known sequences, preferably, within 500 bp from the target locus or more preferably within 200 base pairs of the target locus but in some embodiments may span up to 8 Mbp along a chromosome. In some embodiments, the markers flank the target. FIG. 3. Those of skill in the art will appreciate that two markers flanking the target locus are sufficient enough to identify the presence of the exogenous allele in the haplotype. However, it is within the scope of the disclosure to identify five or more markers unique to the native haplotype. In some embodiments a map is made of markers specific to any haplotype such that the map can be accessed at any time to identify the presence of specific markers on any chromosome of any particular locus of an animal. Once the appropriate markers are identified, methods of determining the presence of those markers can be can be used to either make probes that recognize the desired target by, for example, in situ hybridization with specific probes or make primers that will amplify desired markers, such as by PCR. Other strategies include sequencing a large segment of chromosome comprising the exogenous allele and several markers, confirming the presence of the exogenous DNA, inserted as designed, within the appropriate chromosomal segment. In this case, primers may be designed to flank the target locus in the haplotype. For example, single-molecule real-time sequencing (Pacific Biosciences) can read from 10,000 to 15,000 bp per run while chain termination sequencing (Sanger) can read from about 400 bp to 900 bp. Such distances can be suitable to confirm the correct insertion of, for example the 212 bp POLLED allele properly within the horned haplotype defined at its locus on chromosome 1 of cattle (Bos Taurus).

### Example 5

### Identification of Haplotype Markers Confirming Introgression of Slick Phenotype

“SLICK” is a mutation found in new world cattle including Senepol, Carora, Criollo Limonero and Romosinuano. The term “SLICK” was coined to refer to the cattle's short, glossy coat. This phenotype also includes differences in hair density (less), hair shaft structure type, sweat gland density, average normal body temperature, and thermoregulation efficiency. Cattle having the SLICK phenotype exhibit greatly increased abilities to thermos-regulate in tropical environments and consequently experience considerably less stress in hot environments.

The “SLICK” mutation has been mapped to chromosome 20 of the cattle genome and mutations underlying this phenotype reside with the gene for the prolactin receptor (PRLR). The gene has nine exons that can encode a polypeptide of 581 amino acids. Previous research in Senepol cattle has shown that the phenotype results from a single base deletion in exon 10 (there is no exon 1, recognized exons are 2-10) that introduces a premature stop codon (p.Leu462) and loss of the terminal 120 amino acids from the receptor. This phenotype is referred to herein as SLICK1. Senepol cattle are extremely heat tolerant and have been crossed with many other cattle breeds to provide the benefit of this dominant trait for heat tolerance.

It is now possible to introduce the one or more of the alleles that produce the SLICK phenotype into other cattle without sexual crossing (see, for example, U.S. provisional patent application 62/221,444 to Fahrenkrug et al.). It is therefore important to be able to identify that an animal exhibiting a SLICK phenotype is the product of genetic modification rather than sexual breeding. This is for several reasons. First, most cattle breeds are well characterized and are inbred having a predicted effective population size of between 50 (Holstein/Friesans) and 114 (Braunvieh). The value of such animals is that their characteristics, such as, size, meat production, milk production, number of calves a cow can produce, resistance to disease etc. are well known. Therefore the economic value of the animals is predicated on the animals' ability to match its breed's characteristics. Second, by knowing if an animal shows a trait by virtue of genetic introgression, the animal's genetic history can be followed and confirmed.

Table 1, below provides a marker analysis of SNPs around the SLICK locus. As shown, markers 1-5 are upstream of the SLICK locus on chromosome 20 and markers 6-10 are downstream of the SLICK locus. The row labeled “SNP Allele” is the locus on the chromosome where the markers (SNP) are found naturally in Senepol cattle. The row labeled “Other Allele” is the nucleotide residue of higher minor allele frequency among haired cattle and typically not found in the haplotype linked or containing SLICK. MAF is the frequency of each SNP compared to the WT within an experimental set of genotyped DNAs. The last column shows that the probability of having the SNP allele in the 10 flanking markers and not having the slick mutation is about 8×10-5. However, it should be noted that the sampling of animals for this study was heavily biased toward cattle DNA samples derived from animals influenced by a Criollo genetic base, the native sources of SLICK mutations. Therefore, the frequency of each of the markers is much more prevalent than it would be in any global/random distribution of these markers. The chance that a non-Senepol animal exhibited the deletion at Chr20-39136558 without having any of the linked markers would be 8×10-5 and this value is skewed to be more probable due to the sampling of a heavily influenced Criollo population. As noted in Table 1, the total length of the validation region is 296,033 bp, from 39,047,501 to 39,343,534.

TABLE 1

| Serial Marker | 1 | 2 | 3 | 4 | 5 | Slick |
|---|---|---|---|---|---|---|
| SNP | Chr20- | Chr20- | Chr20- | Chr20- | Chr20- | Chr20- |
| | 39047501 | 39067164 | 39107872 | 39118063 | 39126055 | 39136558 |
| MAF | 0.425 | 0.419 | 0.424 | 0.422 | 0.322 | |
| SNP Allele | G | A | C | G | G | DEL(Slick) |
| Other Allele | T | G | T | A | T | C |
| Slick | 6 | 7 | 8 | 9 | 10 | total = 10 |
| Chr20-39136558 | Chr20- | Chr20- | Chr20- | Chr20- | Chr20- | Prob by |
| | 39179498 | 39179527 | 39235859 | 39343400 | 39343534 | chance |
| | 0.397 | 0.412 | 0.276 | 0.423 | 0.423 | 8.28733E−05 |
| DEL(Slick) | T | G | G | T | T | SLICK |
| | | | | | | Haplotype |
| C | C | C | A | C | C | |

MAF = minor allele frequency; SNP = single nucleotide polymorphism and is denoted by the coordinate position of the SNP on Chr 20 assembly of UMD 3.1 version of the bovine genome. Row designated SNP allele refers to the SNP allele represented in the SLICK Haplotype for the variant derived from Carribbean criollo cattle (i.e. the SLICK causative mutation found in Senepol cattle). Other allele represents the alternative SNP at this position as detected by the marker kit. All SNP listed in this table are bi-allelic. The probability of having the SNP allele in the 10 flanking markers and not having the SLICK mutation is about 8 × 10⁻⁵.

Table 2 identifies the major haplotypes identified by the markers of Table 1.

TABLE 2

| SNP/Marker | Haplotype¹ | Haplotype Count |
|---|---|---|
| SLICK | GACGG-(Del)-TGGTT | 0.541 (n = 915) |
| Seq ID 8 | | |
| WT | TGTAT-C-CCACC | 0.213 (n = 360) |
| Seq ID 9 | | |
| 8 | TGTAT-C-CCGCC | 0.089 (n = 151) |
| Seq ID 10 | | |
| 5 | TGTAG-C-CCACC | 0.029 (n = 49) |
| Seq ID 11 | | |
| 5/8 | TGTAG-C-CCGCC | 0.027 (n = 46) |
| Seq ID 12 | | |
| 5/6/7 | TGTAG-C-TGACC | 0.018 (n = 30) |
| Seq ID 13 | | |
| 8/9/10 | TGTAT-C-CCGTT | 0.018 (n = 22) |
| Seq ID 14 | | |
| | Other Haplotypes (<0.01) | 0.070 (n = 119) |

Seven main haplotypes were identified in the SLICK validation region. As shown in Table 2, the first two haplotypes are SLICK and the WT.

Thus, the identification of reliable markers is a step toward identifying the source of a target sequence (for example, SLICK). In the case of SLICK, there have not been identified any haplotypes having the deletion of the cytosine base that do not also share all the alleles of the SLICK haplotype. Therefore, the chance that an animal from any population would have the cytosine deletion and not have the 10 other markers identified is so exceedingly low as to be impossible.

### Example 6

### Identification of Haplotype Markers for HH1 in Holstein Cattle

The HH1 haplotype on chromosome 5 is associated with reduced conception rate and a deficit of homozygotes in Holstein cattle. A nonsense mutation in APAF1 (APAF1 p.Q579X) was identified within HH1 using whole-genome resequencing of the predicted founder (Chief) and three of his sons. This mutation is predicted to truncate 670 amino acids (53.7) percent of the encoded APAF1 protein that contains a WD40 domain critical to protein-protein interactions. Commercial genotyping of 246,773 Holsteins revealed 5,299 APAF1 heterozygotes and zero homozygotes for the mutation. Recombinant haplotypes, defined as a portion but not all of Chief's HH1 source haplotype, were detected within the pedigree of 78,465 animals that had 54,001 SNP genotypes as of 2011 using findhap.f90 as previously described (VanRaden et al., 2011a; Sonstegard et al., 2013). All copies of the 75-marker source haplotype spanning 7.1 Mbp that contained the putative mutation appeared to trace to Chief and to no other prominent ancestors. Living animals with recombinant haplotypes that are homozygous for only a portion of the source haplotype can rule out that portion of the haplotype as not containing the lethal mutation. After processing all recombinant haplotypes, the area not ruled out was defined as the mutation-critical region, as described by Sonstegard et al. (2013).

Recombination events were detected in 78,465 animals genotyped for 43,385 SNPs from the Illumina BovineSNP50 BeadChips (Illumina, San Diego, Calif.) using edits of Wiggans et al. (2010) and standard output from findhap.f90 (VanRaden et al., 2011a) version 2, which first examined haplotypes of length 600 markers, then 200 markers, and finally output haplotypes of ≦75 markers. The program phases genotypes into haplotypes and detects recombination points between the maternal and paternal haplotype of each genotyped parent. Recombinant haplotypes contain part of the source haplotype and part of a non-source haplotype, and a descendant's phenotype status may be unknown when crossovers occur. Crossovers were detected from genotypes by directly comparing progeny to parent haplotypes within the pedigree. For each crossover, the last marker known to be from the first parental haplotype and the first marker known to be from the second parental haplotype are output. A gap may remain between those two markers if the parental haplotypes are identical in that region, some genotypes are not called, or both parents were heterozygous and alleles could not be phased leading to an unknown crossover location. Because few dams are genotyped, crossovers occurring in maternal ancestors are often undetected (Sonstegard et al., 2013).

Regions homozygous for a section of the source haplotype were removed from consideration of harboring the causative HH1 mutation. For example, if a live animal received the original HH1 haplotype from one parent and the left 20 markers of the HH1 haplotype from the other parent, the region containing those 20 markers was removed from consideration, exactly as described in Sonstegard et al. (2013) for Jersey haplotype 1.

The genomes of Chief, and two of his progeny, Ivanhoe Chief and Valiant were sequenced using sequencing by synthesis chemistry on an Illumina HiSeq 2000 platform (Illumina Inc., San Diego, Calif.). Libraries were prepared from 5 μg of genomic DNA purified from semen straws and data was generated using standard sequencing protocols provided by the manufacturer. Previous sequencing results of Mark (12×) and Chief (6×) using 454 Titanium technology were also used (Larkin et al., 2012).

### Detection of SNPs and Genes

The SNPs in the suspect region of BTA5 were identified using FreeB ayes (Garrison and Marth, 2012). Putative SNPs were accepted if they fit within the following criteria: 4× minimum read coverage with at least two reads aligning in each orientation (forward, reverse), and minimum allele sequencing quality >20. Upon acquiring a list of SNPs in the region, functional annotation of the variants was performed using ANNOVAR (Wang et al., 2010). The ANNOVAR program categorized SNPs by their genic or intergenic locations within the cattle genome. The program reports SNPs located within introns and exons of annotated genes, 5′ and 3′ UTR regions, and those upstream and downstream of gene positions. All coordinates pertaining to SNP and gene positions were converted from Btau4.0 to UMD3.1 genome assemblies using the program LiftOver created by the UCSC Genome Bioinformatics Group (http://genome.ucsc.edu/cgi-bin/hgLiftOver) for consistency with haplotype and genotype datasets.

Animals were selected for validation by querying a large database of 33,415 Holsteins genotyped for 54,001 SNP as constructed previously (Wiggans et al., 2010). Genotype imputation and haplotype frequencies included all 33,415 animals, but the 758 samples selected for further validation were from the Cooperative Dairy DNA Repository, which contains DNA from almost all progeny tested bulls in North America. Haplotype identification was based on the 75 SNP markers designated as the 7.1 Mbp HH1-containing interval on BTA5 (UMD3.1 coordinates 58,638,702 to 65/743,920; VanRaden et al., 2011b). An additional query was implemented to select a diverse set of non-carriers that had unique heterozygous haplotype combinations in this interval.

An SNP genotyping panel (Sequenom Inc., San Diego, Calif.) designed for the validation test (Page et al., 2004) was composed of 24 bi-directional assays for 12 putative SNPs in the refined HH1 interval region. This included all SNPs with gene boundaries found within this interval, as well as five additional SNPs observed near adjacent genes in the interval or in distal flanking regions from the APAF1 stop-gain mutation. A total of 22 of the 24 SNP assays were functional; one SNP locus was monomorphic. The call rate for all SNP loci was 100% except for UMD3_63107293 (99.3%) and UMD3_62591311 (99.9%). Results from the bi-directional assays for each SNP locus were compared for concordance and integrated into a single marker genotype score for each animal across the 11 SNP loci. Haplotypes of 11 informative SNPs were determined by PHASE v2.1.1 (Stephens et al., 2001), and a total of 24 probable haplotypes were identified (Table 3). These haplotypes are much shorter and different than those originally defined by the 75-marker window (derived from the 54,001 chip) spanning 7.1 Mbp that was used to find HH1. Two different numbering systems exist: one for the more than 2,000 different haplotypes in this 7.1 Mbp window, and a second for the 24 haplotypes in the narrow 11 SNP window for validation (Table 3).

TABLE 3
Table 3: Haplotypes of the 11 informative SNPs in Holstein Haplotype
(HH1) validation region. Allele calls were designated either 0 or 1 -
meaning that genome reference allele is designated 0 and presence of
an alternative allele is designated 1.

| Haplotype¹ | Haplotype Count |
|---|---|
| 000010-0-0011 | 7 |
| 000011-0-0001 | 5 |
| 000011-0-0111 | 9 |
| 000011-0-1000 | 64 |
| 000011-0-1001 | 243 |
| 000110-0-0001 | 60 |
| 011010-0-1001 | 1 |
| 011110-0-0001 | 4 |
| 011110-0-0011 | 21 |
| 011110-0-0111 | 179 |
| 011110-0-1000 | 1 |
| 011110-0-1001 | 25 |
| 011100-0-0111 | 9 |
| 100010-0-0011 | 7 |
| 100010-0-1001 | 4 |
| 100011-0-0111 | 22 |
| 100011-0-1001 | 82 |
| 100110-0-0001 | 69 |
| 111110-0-0001 | 1 |
| 111110-0-0011 | 34 |
| 111110-0-0110 | 1 |
| 111110-0-0111 | 117 |
| 111110-0-1001 | 53 |
| 111100-1-0111 | 498 |

¹APAF1 stop-gain mutation is the 7th marker of this haplotype, and the only haplotype carrying the APAF1 marker. Animals homozygous 1 at this site are lost in utero. Notice there is only one haplotype in this marker kit that designates the animals carrying the lethal mutation at marker 7 (111100-1-0111). Any animals with the zero allele at this position and this haplotype would have to be the result of genome editing. HH1 information is found at this URL - http://omia.angis.org.au/OMIA000001/9913/. This mutation is found only in Holstein cattle, so it only would be edited to wild type allele in Holstein cattle. SNP positions on Chr 5 - UMD3_62591311, UMD3_63051612, UMD3_63052631, UMD3_63088973, UMD3_63091578, UMD3_63107293, UMD3_63150400, UMD3_63198664, UMD3_63209396, UMD3_63228106, UMD3_63486133.

Following validation testing using multiple datasets with varying markers and animals (Adams et al.; J. Dairy Sci, J Dairy Sci. 2016 August; 99(8):6693-701), a test for the stop-gain mutation was added to the GeneSeek Genomic Profiler (GGP) BeadChip (GeneSeek-Neogen, Lincoln, Nebr.; Neogen Corp., 2013) and subsequent chips, and genotypes were received for 246,773 Holsteins as part of routine genomic predictions.

As illustrated in Table 3, any animals with the zero allele at the 7^{th} marker, (UMD3_63150400) and that has the 111100-X-0111 haplotype would have to be the result of genome editing. Further, the possibility that the haplotype could have a random mutation at the 7^{th} marker only is 1 in 2.85 billion. Thereby obviating any possibility that the presence of a zero at the seventh marker could be a random occurrence.

While this disclosure has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this disclosure, as set forth above, are intended to be illustrative not limiting. Various changes may be made without departing from the spirit and scope of the disclosure. Therefore, the disclosure is intended to embrace all known or later-developed alternatives, modifications, variations, improvements and/or substantial equivalents of these exemplary embodiments.

The following paragraphs enumerated consecutively from 1 through 43 provide for various additional aspects of the present disclosure. In one embodiment, in a first paragraph, (1), the present disclosure provides:

1. A process of making a kit for testing a livestock animal to identify an exogenous allele in a native haplotype comprising:

identifying a native haplotype potentially including the exogenous allele;

identifying the exogenous allele;

preparing two or more probes specific to the haplotype.

2. The process of paragraph 1, wherein identifying the haplotype comprises detecting the presence of markers native to the livestock in the haplotype.
3. The process of paragraphs 1 and 2, wherein identifying the exogenous allele comprises identifying the presence of a foreign allele within the haplotype.
4. The process of paragraphs 1 through 3, wherein the exogenous allele is introduced by non-meiotic introgression.
5. The process of paragraphs 1 through 4, wherein the desired haplotype is determined by two or more markers.
6. The process of paragraphs 1 through 5 wherein the two or more markers are present on either side of the exogenous allele.
7. The process of paragraphs 1 through 6, wherein the two or more markers are found within 2 Mb of the allele.
8. The process of paragraphs 1 through 7, wherein the two or more markers are found within 1 Mb of the allele.
9. The process of paragraphs 1 through 8, wherein detecting the presence of markers native to the livestock comprises a probe specific to each marker.
10. The process of paragraphs 1 through 9, wherein the markers are selected from Bos Taurus and comprise: Chr20-39047501, Chr20-39067164, Chr20-39107872, Chr20-39118063, Chr20-39126055, Chr20-39136558, Chr20-39179498, Chr20-39179527, Chr20-39235859, Chr20-39343400, Chr20-39343534, Chr5-UMD3_62591311, Chr5-UMD3_63051612, Chr5-UMD3_63052631, Chr5-UMD3_63088973, Chr5-UMD3_63091578, Chr5-UMD3_63107293, Chr5-UMD3_63150400, Chr5-UMD3_63198664, Chr5-UMD3_63209396, Chr5-UMD3_63228106, Chr5-UMD3_63486133.
11. The process of paragraphs 1 through 10, wherein the probe is used in PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).
12. The process of paragraphs 1 through 11, wherein the markers are sequence specific regions of the haplotype.
13. The process of paragraphs 1 through 12, wherein the exogenous allele is derived from a different lineage, breed or species from the livestock.
14. The process of paragraph 1 through 13, wherein identifying the exogenous allele comprises using a probe specific to the exogenous allele.
15. The process of paragraphs 1 through 14, wherein the probe is used in PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).
16. The process of paragraphs 1 through 15, wherein the exogenous allele comprises at least one base foreign to the livestock's native allele.
17. A method of identifying the presence of an exogenous, target, allele in a livestock animal comprising:

i) identifying a native haplotype in the livestock animal;

ii) identifying an allele exogenous to the haplotype;

iii) determining the presence of the exogenous allele in the haplotype.

18. The method of paragraphs 1 through 17, wherein identifying the desired haplotype comprises detecting the presence of markers native to the livestock in the haplotype.
19. The method of any of paragraphs 1 through 18, wherein identifying the exogenous allele in the haplotype comprises identifying the presence of a foreign allele within the haplotype.
20. The method of any of paragraphs 1 through 19, wherein the exogenous allele is introduced by non-meiotic introgression.
21. The method of any of paragraphs 1 through 20, wherein the haplotype is identified by two or more markers.
22. The method of any of paragraphs 1 through 21, wherein the two or more markers are present on either side of the exogenous allele.
23. The method of any of paragraphs 1 through 22, wherein the two or more markers are identified using probes specific to each marker.
24. The method of any of paragraphs 1 through 23, wherein the two or more markers are found within 2 MB of the allele.
25. The method of any of paragraphs 1 through 24, wherein the two or more markers are found within 1 MB of the allele.
26. The method of any of paragraphs 1 through 25, wherein the markers are selected from Bos Taurus and comprise: Chr20-39047501, Chr20-39067164, Chr20-39107872, Chr20-39118063, Chr20-39126055, Chr20-39136558, Chr20-39179498, Chr20-39179527, Chr20-39235859, Chr20-39343400, Chr20-39343534, Chr5-UMD3_62591311, Chr5-UMD3_63051612, Chr5-UMD3_63052631, Chr5-UMD3_63088973, Chr5-UMD3_63091578, Chr5-UMD3_63107293, Chr5-UMD3_63150400, Chr5-UMD3_63198664, Chr5-UMD3_63209396, Chr5-UMD3_63228106, Chr5-UMD3_63486133
27. The method of any of paragraphs 1 through 26, wherein detecting the presence of markers native to the livestock comprises a probe specific to each marker.
28. The method of any of paragraphs 1 through 27, wherein the probe can be used in PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).
29. The method of any of paragraphs 1 through 28, wherein the markers are sequence specific regions of the haplotype.
30. The method of any of paragraphs 1 through 29, wherein the exogenous allele is derived from a different lineage, breed or species from the livestock.
31. The method of any of paragraphs 1 through 30, wherein identifying the exogenous allele comprises using a probe specific to the exogenous allele.
32. The method of any of paragraphs 1 through 31, wherein the probe is used in PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).
33. The method of any of paragraphs 1 through 32, wherein the exogenous allele comprises at least one base foreign to the livestock's native haplotype.
34. A kit for determining the presence of an exogenous allele introduced into a haplotype using non-meiotic introgression comprising:

a probe specific to an allele foreign to an animal

two or more probes specific to a haplotype of the animal

35. The kit of paragraph 34, further comprising instructions for use.
36. The kit of paragraphs 34 and 35, further comprising a container for holding reaction mixtures.
37. The kit of paragraphs 34 through 36, further comprising reagents.
38. The kit of paragraphs 34 through 38, wherein the probes are for use in: PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).
39. The kit of paragraphs 34 through 38, wherein the probes are specific for markers comprising: Chr20-39047501, Chr20-39067164, Chr20-39107872, Chr20-39118063, Chr20-39126055, Chr20-39136558, Chr20-39179498, Chr20-39179527, Chr20-39235859, Chr20-39343400, Chr20-39343534, Chr5-UMD3_62591311, Chr5-UMD3_63051612, Chr5-UMD3_63052631, Chr5-UMD3_63088973, Chr5-UMD3_63091578, Chr5-UMD3_63107293, Chr5-UMD3_63150400, Chr5-UMD3_63198664, Chr5-UMD3_63209396, Chr5-UMD3_63228106, Chr5-UMD3_63486133.
40. A genetically modified animal consisting of a genome edited at about Chr5-UMD3_63150400 or about Chr20-39136558 of Bos Taurus.
41. A method of making a genetically modified animal having a slick phenotype comprising a modification at about the Chr20-39136558 locus of Bos Taurus.
42. An in vitro animal cell comprising a modification at about Chr5-UMD3_63150400 or about Chr20-39136558 of Bos Taurus.
43. Use of a method according to any of the above paragraphs for detecting the presence of a foreign allele in a desired haplotype.

All patents, publications, and journal articles set forth herein are hereby incorporated by reference herein; in case of conflict, the instant specification is controlling.

While this disclosure has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to this disclosure, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents of these exemplary embodiments.

1. A process of making a kit for testing a livestock animal to identify an exogenous allele in a native haplotype comprising:
i) identifying a native haplotype potentially including the exogenous allele;
ii) identifying the exogenous allele;
iii) preparing two or more probes specific to the haplotype.

2. The process of claim 1, wherein identifying the haplotype comprises detecting the presence of markers native to the livestock in the haplotype.

3. The process of claim 1, wherein identifying the exogenous allele comprises identifying the presence of a foreign allele within the haplotype.

4. The process of claim 1, wherein the exogenous allele is introduced by non-meiotic introgression.

5. The process of claim 1, wherein the desired haplotype is determined by two or more markers.

6. The process of claim 5 wherein the two or more markers are present on either side of the exogenous allele.

7. The process of claim 5, wherein the two or more markers are found within 2 Mb of the allele.

8. The process of claim 5, wherein the two or more markers are found within 1 Mb of the allele.

9. The process of claim 2, wherein detecting the presence of markers native to the livestock comprises a probe specific to each marker.

10. The process of claim 2, wherein the markers are selected from Bos Taurus and comprise: Chr20-39047501, Chr20-39067164, Chr20-39107872, Chr20-39118063, Chr20-39126055, Chr20-39136558, Chr20-39179498, Chr20-39179527, Chr20-39235859, Chr20-39343400, Chr20-39343534, Chr5-UMD3_62591311, Chr5-UMD3_63051612, Chr5-UMD3_63052631, Chr5-UMD3_63088973, Chr5-UMD3_63091578, Chr5-UMD3_63107293, Chr5-UMD3_63150400, Chr5-UMD3_63198664, Chr5-UMD3_63209396, Chr5-UMD3_63228106, Chr5-UMD3_63486133.

11. The process of claim 9, wherein the probe is used in PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).

12. The process of claim 5, wherein the markers are sequence specific regions of the haplotype.

13. The process of claim 1, wherein the exogenous allele is derived from a different lineage, breed or species from the native allele.

14. The process of claim 1, wherein identifying the exogenous allele comprises using a probe specific to the exogenous allele.

15. The process of claim 14, wherein the probe is used in PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).

16. The process of claim 12, wherein the exogenous allele comprises at least one base foreign to the livestock's native allele.

17. A method of identifying the presence of an exogenous, target, allele in a livestock animal comprising:
i) identifying a native haplotype in the livestock animal;
ii) identifying an allele exogenous to the haplotype;
iii) determining the presence of the exogenous allele within the haplotype.

18. The method of claim 17, wherein identifying the desired haplotype comprises detecting the presence of markers native to the livestock in the haplotype.

19. The method of claim 18, wherein identifying the exogenous allele in the haplotype comprises identifying the presence of a foreign allele within the haplotype.

20. The method of claim 17, wherein the exogenous allele is introduced by non-meiotic introgression.

21. The method of claim 17, wherein the haplotype is identified by two or more markers.

22. The method of claim 21, wherein the two or more markers are present on either side of the exogenous allele.

23. The method of claim 21, wherein the two or more markers are identified using probes specific to each marker.

24. The method of claim 21, wherein the two or more markers are found within 2 MB of the allele.

25. The method of claim 21, wherein the two or more markers are found within 1 MB of the allele.

26. The method of claim 21, wherein the markers are selected from Bos Taurus and comprise: Chr20-39047501, Chr20-39067164, Chr20-39107872, Chr20-39118063, Chr20-39126055, Chr20-39136558, Chr20-39179498, Chr20-39179527, Chr20-39235859, Chr20-39343400, Chr20-39343534, Chr5-UMD3_62591311, Chr5-UMD3_63051612, Chr5-UMD3_63052631, Chr5-UMD3_63088973, Chr5-UMD3_63091578, Chr5-UMD3_63107293, Chr5-UMD3_63150400, Chr5-UMD3_63198664, Chr5-UMD3_63209396, Chr5-UMD3_63228106, Chr5-UMD3_63486133.

27. The method of claim 19, wherein detecting the presence of markers native to the livestock comprises a probe specific to each marker.

28. The method of claim 23, wherein the probe can be used in PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).

29. The method of claim 21, wherein the markers are sequence specific regions of the haplotype.

30. The method of claim 17, wherein the exogenous allele is derived from a different lineage, breed or species from the livestock.

31. The method of claim 17, wherein identifying the exogenous allele comprises using a probe specific to the exogenous allele.

32. The method of claim 30, wherein the probe is used in PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).

33. The method of claim 17, wherein the exogenous allele comprises at least one base foreign to the livestock's native haplotype.

34. A kit for determining the presence of an exogenous allele introduced into a haplotype using non-meiotic interogression comprising:
i) a probe specific to an allele foreign to an animal
ii) two or more probes specific to a haplotype of the animal

35. The kit of claim 34, further comprising instructions for use.

36. The kit of claim 34, further comprising a container for holding reaction mixtures.

37. The kit of claim 34, further comprising reagents.

38. The kit of claim 34, wherein the probes are for use in: PCR, array-based assays, high resolution melting (HRM) analysis, fragment analysis, Sanger fragment analysis, amplified fragment length polymorphism (AFLP) analysis, restriction fragment length polymorphism (RFLP) analysis, or single strand conformation polymorphism analysis (SSCP).

39. The kit of claim 34 wherein the probes are specific for markers comprising: Chr20-39047501, Chr20-39067164, Chr20-39107872, Chr20-39118063, Chr20-39126055, Chr20-39136558, Chr20-39179498, Chr20-39179527, Chr20-39235859, Chr20-39343400, Chr20-39343534, Chr5-UMD3_62591311, Chr5-UMD3_63051612, Chr5-UMD3_63052631, Chr5-UMD3_63088973, Chr5-UMD3_63091578, Chr5-UMD3_63107293, Chr5-UMD3_63150400, Chr5-UMD3_63198664, Chr5-UMD3_63209396, Chr5-UMD3_63228106, Chr5-UMD3_63486133.

40. A genetically modified animal consisting of a genome edited at about Chr5-UMD3_63150400 or about Chr20-39136558 of Bos Taurus.

41. A method of making a genetically modified animal having a slick phenotype comprising a modification at about the Chr20-39136558 locus of Bos Taurus.

42. An in vitro animal cell comprising a modification at about Chr5-UMD3_63150400 or about Chr20-39136558 of Bos Taurus.
